(19) United States
(12) Patent Application Publication
(10) Pub. No.: US 2013/0302841 A1
(43) Pub. Date: Nov. 14, 2013

(54) PROGNOSIS AND RISK ASSESSMENT OF PATIENTS WITH NON-SPECIFIC COMPLAINTS

(71) Applicants: Joachim Struck, Berlin (DE); Sven Giersdorf, Berlin (DE); Oliver Hartmann, Berlin (DE); Roland Bingisser, Basel (CH); Christian Nickel, Basel (CH)

(72) Inventors: Joachim Struck, Berlin (DE); Sven Giersdorf, Berlin (DE); Oliver Hartmann, Berlin (DE); Roland Bingisser, Basel (CH); Christian Nickel, Basel (CH)

(73) Assignee: B.R.A.H.M.S GMBH, Hennigsdorf (DE)

(21) Appl. No.: 13/882,895

(22) PCT Filed: Oct. 31, 2011
(86) PCT No.: PCT/EP2011/069170

§ 371 Date: Jul. 29, 2013

(30) Foreign Application Priority Data
Nov. 1, 2010 (EP) 10189598.5

(51) Int. Cl. G01N 33/68

(52) U.S. Cl. CPC G01N 33/6893
USPC 435/28; 436/501; 435/39

(57) ABSTRACT

The present invention relates to the determination of the level of marker peptides in a sample derived from a bodily fluid of a subject presenting to the emergency department with non-specific complaints.

### FIELD OF THE INVENTION

The present invention is in the field of clinical diagnostics. Particularly, the present invention relates to the determination of the level of marker peptides in a sample derived from a bodily fluid of a subject presenting to the emergency department with non-specific complaints.

### BACKGROUND OF THE INVENTION

Patients presenting to emergency departments (ED) with non-specific complaints (NSC) are a well-known but poorly defined population. Affected individuals often complain of “not feeling well”, “feeling weak”, “being tired”, feeling “dizzy”, or simply complain of being unable to cope with usual daily activities (van Bokhoven et al. 2008. J Clin Epidemiol; 61:318-22). Some patients may fail to recall why they were sent to the ED. During the care of NSC patients, ED physicians face a broad differential diagnosis ranging from insufficient home care to acute life-threatening conditions (Gordon 1986. Geriatrics 41(4): 75-80). Patients with NSC are among the most challenging to ED physicians (Chew and Birnbaumer 1999. Emerg Med Clin North Am 17(1):265-78). Moreover, the clinical picture is often blurred by factors such as co-morbidities, poly-pharmacy or altered mental state.

Vanpee et al. demonstrated that up to 20% of older individuals presenting to the ED have no specific complaints (Vanpee et al. 2001. Eur J Emerg Med 8(4):301-4). Furthermore, 50% of older individuals without specific complaints suffered from an acute medical problem (Rutschmann et al. 2005. Swiss Med Wkly 135(9-10):145-50). The ED geriatric population is a particularly high risk group for adverse outcomes (e.g. functional decline, dependence, and death) (McCusker et al. 2001. J Am Geriatr Soc 49(10):1272-81). The needs of these patients with NSC must be better defined and development of efficient and safe decision and management strategies is essential (Vanpee et al. 2001. Eur J Emerg Med 8(4):301-4; Rutschmann et al. 2005. Swiss Med Wkly 135(9-10):145-50; Sanders A B. Emergency Care of the Elder Person. St. Louis: Beverly Cracom Publications ed. 1996).

Patient management in emergency medicine often relies on diagnostic and treatment protocols based on presenting chief complaints such as for acute chest pain, dyspnoea or flank pain (Lee and Goldman 2000. N Engl J Med 342(16):1187-95; Gupta et al. 2002. Ann Emerg Med 40(2):180-6; American Thoracic Society 1999. Am J Respir Crit. Care Med 1590:321-40; Wang et al. 2005. JAMA 294(15):1944-56; Kartal et al. 2006. Emerg Med J 23(5):341-4; European Curriculum for Emergency Medicine. A document of the EuSEM Task Force on Curriculum approved by the Council of the European Society for Emergency Medicine and by the UEMS Multidisciplinary Joint Committee on Emergency Medicine. 2008). No comparable management protocols have been published for NSC—most likely due to a lack of a consistent definition for NSC and the paucity of research regarding differential diagnosis and efficient work-up strategies in this population (Rosendal et al. 2005. BMJ 330(7481):4-5). Uncertainty often accompanies the diagnostic process with potentially superfluous confirmatory testing in order to exclude an underlying serious condition (Vanpee et al. 2001. Eur J Emerg Med 8(4):30′-4; Sanders A B. Emergency Care of the Elder Person. St. Louis: Beverly Cracom Publications ed. 1996). This may result in prolonged waiting times, ineffective triage and inadequate referrals (Sanders 1992. Ann Emerg Med 21(7):830-4; Knottnerus et al. 1986. Ned Tijdschr Geneeskd 130(9):402-5).

Nemec et al. provided a stringent definition of non-specific complaints, that is included here by reference (Nemec et al. 2010. Acad Emerg Med 17(3):284-292).

It was surprisingly found that the marker peptides or fragments thereof or precursors or fragments thereof, are independent predictors for serious conditions including death and hospitalisation in patients presenting to emergency departments with non-specific complaints.

### SUMMARY OF THE INVENTION

Subject of the invention is a method for the prediction and risk assessment of patients with non-specific complaints comprising the determination of marker peptides or fragments thereof or precursors or fragments thereof with at least 12 amino acids in a sample taken from said subject.

### DETAILED DESCRIPTION OF THE INVENTION

Subject of the invention is a method for the prediction and risk assessment of patients with non-specific complaints comprising the determination of marker peptides or fragments thereof or precursors or fragments thereof with at least 12 amino acids in a sample taken from said subject.

Subject of the present invention is a method for the risk assessment or the prognosis of an outcome or the stratification of patients with non-specific complaints,
the method comprising the steps of:
   providing a sample from a bodily fluid from said patient,
   determining in the sample the level of a marker peptide selected from the group of proANP, proBNP, proAVP, proADM, proET-1, PCT, PRX-4 or fragments thereof comprising at least 12 amino acids in length, and
   correlating said marker peptide level to the risk of acquiring a serious condition and/or death or to the prognosis of getting a serious condition and/or death in a patient with non-specific complaints.

In one embodiment non-specific complaints are defined as the entity of complaints not part of the set of specific complaints for which evidence-based management protocols for emergency physicians (EPs) exist.

Serious conditions may be defined as potentially life-threatening or requiring early intervention to prevent health status deterioration.

In a preferred embodiment of the method according to invention the risk assessment or the prognosis or the stratification relates to the risk of getting a serious condition including death or patients are stratified into either a group of patients likely getting a serious condition and/or death or into a group of patients which do not likely get a serious condition including death. The stratification of patients may be a stratification according to the severity of their condition into either a group of patients likely getting a serious condition and/or death or into a group of patients which do not likely get a serious condition including death.

In one preferred embodiment the patient has not a primary disease which has been diagnosed before. This means that the patient has been considered as being healthy before said non-specific complaints occurred.

In another preferred embodiment the patient has a primary disease when said non-specific complaints occur, possibly an already diagnosed primary disease. The determination of the level of a marker peptide selected from the group of proANP, proBNP, proAVP, proADM, proET-1, PCT, PRX-4 or fragments thereof comprising at least 12 amino acids in length enables the risk assessment or the prognosis of an outcome or the stratification of patients with non-specific complaints by correlating said level to the risk of acquiring a serious condition and/or death or to the prognosis of getting a serious condition and/or death in a patient with non-specific complaints wherein this serious condition and/or death may be either related to (a) said (diagnosed) primary disease or to (b) a second further disease which may have been yet diagnosed or undiagnosed. These non-specific complaints may be regarded as early, although non-specific, symptoms related to said primary disease or early, although non-specific, symptoms of a second further disease which has been already acquired.

One patient with non-specific complaints may present with several co-morbid illnesses. Patients may thus present with multi-morbidity, thus, having more than one co-morbidity. Some patients included in the study presented with up to 11 co-morbidities including chronic hypertension, coronary artery disease, dementia, diabetes, cerebrovascular disease, chronic alcoholism, depression, COPD, any solid tumor, chronic heart failure, leukemia, falls within the last 3 months, any psychiatric disorder.

“Serious condition” in the context of the present invention is defined as any potentially life-threatening condition (e.g. myocardial infarction) or any condition which requires an early intervention to prevent health status deterioration leading to possible morbidity, disability or death (e.g. severe hyponatremia). Obviously, the natural course of an underlying serious condition according to this definition should not be awaited. Moreover, any death occurring condition after a defined time, e.g. after 3 days, 5 days, 7 days, 10 days, 14 days, 20 days, 3 weeks, 4 weeks, 30 days, 45 days, 60 days, 90 days, 3 months, 6 months, 1 year, of the initial ED presentation is judged as a serious condition.

The term “outcome” herein relates for instance to the survival of a patient or the occurrence of a serious condition including death after a defined time, e.g. after 3 days, 5 days, 7 days, 10 days, 14 days, 20 days, 3 weeks, 4 weeks, 30 days, 45 days, 60 days, 90 days, 3 months, 6 months, 1 year.

According to the method, a patient with non-specific complaints is attributed to a risk of getting a serious condition including death upon presentation to the ED within 1 year, more preferred within 6 months, even more preferred within 3 months, even more preferred within 60 days, most preferred within 30 days.

In the present invention, the term “prognosis” denotes a prediction of how a subject's (e.g. a patient's) medical condition will progress. This may include an estimation of the chance of recovery or the chance of an adverse outcome for said subject.

In the present invention, the term “risk assessment” denotes an assignment of a probability to experience certain adverse events to an individual. Hereby, the individual may preferably be accounted to a certain risk category, wherein categories comprise for instance high risk versus low risk, or risk categories based on numeral values, such as risk category 1, 2, 3, etc.

In a preferred embodiment the serious condition is selected from the group comprising death, hospitalisation or admission to ICU.

As mentioned herein in the context of marker peptides and precursors thereof the term “fragment” refers to smaller proteins or peptides derivable from larger proteins or peptides, which hence comprise a partial sequence of the larger protein or peptide. Said fragments are derivable from the larger proteins or peptides by saponification of one or more of its peptide bonds. “Fragments” of the marker peptides proANP, proBNP, proET-1, proADM, proAVP, Peroxiredoxin-4 and PCT preferably relate to fragments of at least 12 amino acids in length. Such fragments are preferably detectable with immunological assays as described herein.

The sequence of the 153 amino acid pre-pro-ANP is shown in SEQ ID NO:1. Upon cleavage of an N-terminal signal peptide (25 amino acids) and the two C-terminal amino acids proANP (SEQ ID NO:2) is released. This prohormone is cleaved into the mature 28 amino acid peptide ANP, also known as ANP (1-28) or α-ANP, and the amino terminal fragment ANP (1-98) (NT-proANP, SEQ ID NO:3). Mid-regional proANP (MR-proANP) is defined as NT-proANP or any fragments thereof comprising at least amino acid residues 53-90 (SEQ ID NO:4) of proANP.

In a preferred embodiment of the method according to the invention the level of the proANP precursor fragment, MR-proANP, is determined.

The amino acid sequence of the precursor peptide of Adrenomedulin (pre-pro-Adrenomedullin) is given in SEQ ID NO:5. Pro-Adrenomedullin relates to amino acid residues 22 to 185 of the sequence of pre-pro-Adrenomedullin. The amino acid sequence of pro-Adrenomedullin (pro-ADM) is given in SEQ ID NO:6. MR-pro-Adrenomedullin (MR-pro-ADM) relates to amino acid residues 45-92 of pre-pro-ADM. The amino acid sequence of MR-pro-ADM is provided in SEQ ID NO:7.

In another preferred embodiment of the method according to the invention the level of the proADM precursor fragment, MR-proADM, is determined.

The sequence of the 164 amino acid precursor peptide of Vasopressin (pre-pro-Vasopressin) is given in SEQ ID NO:8. Pro-Vasopressin relates to the amino acid residues 29 to 164 of the sequence of pre-pro-Vasopressin. The amino acid sequence of pro-Vasopressin is given in SEQ ID NO:9. Pro-Vasopressin is cleaved into mature Vasopressin, Neurophysin II and C-terminal proVasopressin (CT-proAVP or Copeptin). Coeptin relates to amino acid residues 126 to 164 of pre-pro-Vasopressin. The amino acid sequence of Copeptin is provided in SEQ ID NO:10. Neurophysin II comprises the amino acid residues 32 to 124 of pre-pro-Vasopressin and its sequence is shown in SEQ ID NO:11.

In another preferred embodiment of the method according to the invention the level of the proAVP precursor fragment, Copeptin, is determined.

The sequence of the 212 amino acid precursor peptide of Endothelin-1 (pre-pro-Endothelin-1) is given in SEQ ID NO:12. Pro-ET-I relates to the amino acid residues 18 to 212 of the sequence of pre-pro-ET-1. The amino acid sequence of pro-ET-1 is given in SEQ ID NO:13. Pro-ET-1 is cleaved into mature ET-1, big-ET-1 and C-terminal proET-1 (CT-proET-1). CT-proET-1 relates to amino acid residues 168 to 212 of pre-pro-ET-1. The amino acid sequence of CT-proET-1 is provided in SEQ ID NO:14.

In another preferred embodiment of the method according to the invention the level of the proET-1 precursor fragment, CT-proET-1, is determined.

The sequence of the 134 amino acid precursor peptide of brain natriuretic peptide (pre-pro-BNP) is given in SEQ ID NO:15. Pro-BNP relates to amino acid residues 27 to 134 of pro-pro-BNP. The sequence of pro-BNP is shown in SEQ ID NO:16. Pro-BNP is cleaved into N-terminal pro-BNP (NT-pro-BNP) and mature BNP. NT-pro-BNP comprises the amino acid residues 27 to 102 and its sequence is shown in SEQ ID NO:17. The SEQ ID NO:18 shows the sequence of BNP comprising the amino acid residues 103 to 134 of the pre-pro-BNP peptide.

In another preferred embodiment of the method according to the invention the level of the proBNP precursor fragment, NT-proBNP, is determined.

Procalcitonin is a precursor of calcitonin and katacalcin. The amino acid sequence of PCT 1-116 is given in SEQ ID NO:19.

In another preferred embodiment of the method according to the invention the level of PCT consisting of amino acids 1 to 116 or 2 to 116 or 3 to 116 is determined.

The amino acid sequence of PRX-4 is set forth in SEQ ID NO:20. The determination of PRX-4 encompasses the determination of PRX-4 and/or a homomultimer of PRX-4 and/or a heteromultimer of PRX-4 with one or more other peroxiredoxins and/or a fragment of PRX-4.

In another preferred embodiment of the method according to the invention the level of a marker peptide selected from the group of proANP, proBNP, proAVP, proADM, proET-1, PRX-4 or fragments thereof comprising at least 12 amino acids in length is determined in said sample.

In another preferred embodiment of the method according to the invention the level of at least two marker peptides selected from the group comprising MR-proANP, Copeptin, MR-proADM, CT-proET-1, PRX-4, NT-proBNP and PCT are determined.

In yet another preferred embodiment of the method according to the invention the level of Copeptin and PRX-4 are determined.

In yet another preferred embodiment of the method according to the invention the level of at least one marker peptide selected from the group comprising MR-proANP, Copeptin, MR-proADM, CT-proET-1, PRX-4, NT-proBNP and PCT is determined and combined with one or more clinical or laboratory parameter or patients characteristics selected from the group comprising C-reactive protein (CRP), creatinine, albumin, urea, glomerular filtration rate (GFR), white blood cell count (WBC), troponin, myeloperoxidase, neopterin, GDF-15, ST2, cystatin-C, Charlson Comorbidity Index (CCI), Katz ADL, age and gender.

In yet another preferred embodiment of the method according to the invention the level of Copeptin and PRX-4 are determined and combined with age and/or gender.

In yet another preferred embodiment of the method according to the invention the level of Copeptin and PRX-4 are determined and combined with Charlson Comorbidity Index.

In yet another preferred embodiment of the method according to the invention the level of Copeptin and PRX-4 are determined and combined with Katz ADL.

According to one preferred embodiment of the method according to the present invention patient stratification relates to the management of a patient including the decision for admission to hospital or intensive care unit, the decision for relocation of the patient to a specialized hospital or a specialized hospital unit, the decision for relocation of the patient to a specialized hospital or a specialized hospital unit, the evaluation for an early discharge from the intensive care unit or hospital or the allocation of resources (e.g. physician and/or nursing staff, diagnostics, therapeutics).

Chanson Comorbidity Index (CCI) predicts the one-year mortality for a patient who may have a range of co-morbid conditions such as heart disease, AIDS, or cancer (a total of 22 conditions) (Charlson et al. 1987. J Chronic Dis 40(5):373-83). Each condition is assigned with a score of 1, 2, 3 or 6 depending on the risk of dying associated with this condition. Then the scores are summed up and given a total score which predicts mortality.

The Katz Index of Independence in Activities of Daily Living, commonly referred to as the Katz index or Katz ADL, is the most appropriate instrument to assess functional status as a measurement of the patients ability to perform activities of daily living independently. Clinicians typically use the tool to detect problems in performing activities of daily living and to plan care accordingly. The index ranks adequacy of performance in the six functions of bathing, dressing, toileting, transferring, continence, and feeding. Patients are scored yes/no for independence in each of the six functions. A score of 6 indicates full function, 4 indicates moderate impairment, and 2 or less indicates severe functional impairment (Katz and Akpom 1976. Med Care 14(5 Suppl):116-8; Katz et al. 1970. Gerontologist 10:20-30).

The term “correlating”, as used herein in reference to the use of diagnostic and prognostic marker(s), refers to comparing the presence or amount of the marker(s) in a patient to its presence or amount in persons known to suffer from, or known to be at risk of, a given condition. A marker level in a patient sample can be compared to a level known to be associated with a specific diagnosis. The sample's marker level is said to have been correlated with a diagnosis; that is, the skilled artisan can use the marker level to determine whether the patient suffers from a specific type diagnosis, and respond accordingly. Alternatively, the sample's marker level can be compared to a marker level known to be associated with a good outcome (e.g. the absence of disease etc.). In preferred embodiments, a panel of marker levels is correlated to a global probability or a particular outcome.

The term “level” in the context of the present invention relates to the concentration (preferably expressed as weight/volume; w/v) of marker peptides taken from a sample of a patient.

### Definition of Non-Specific Complaints

A specific chief complaint usually provides key information and allows generating a working diagnosis or following a predefined diagnostic protocol. Specific complaints are well-recognized as such in the literature and diagnostic protocols are often applied (Marx J A Hockberger R, Walls R. Rosen's Emergency Medicine: Concepts and Clinical. Sixth Edition ed. St Louis: Mosby; 2005; Siegenthaler W. Differential Diagnosis in Internal Medicine: From Symptom to Diagnosis. New York: Thieme Medical Publishers; 2007).

In contrast to specific complaints, we defined NSC as the entity of all complaints which are not part of the set of specific complaints or signs or where an initial working diagnosis cannot be established. It is necessary to define NSC as the remainder after exclusion of specific complaints, because an active definition would require an almost endless enumeration of possible non-specific complaints. Such a long and complicated definition would likely exclude certain NSC patients because their symptoms failed to exactly match the predefined list. We use the term working diagnosis in the context of our NSC definition for situations where patients present with NSC, but a diagnosis is likely nevertheless given the facts and findings at the time of presentation.

FIG. 1 summarizes this definition in a procedural way. In the most preferred embodiment nonspecific complaints are defined as complaints which lead to an inclusion according to FIG. 1. This means that the patient according to the invention does not exhibit one of the following complaints: pain (chest, abdominal, head, leg, joint, back), dyspnea, cough, weakness (localized), stroke-like symptoms, swollen extremity (leg, arm), diarrhea, dysuria, GCS <14, confusion, intoxication, seizure, bleeding, syncope, anxiety, psychotic symptoms, suicidal ideation, skin lesion, allergic skin reaction, fever, vertigo, palpitations, nausea with vomiting, trauma. Furthermore, the question whether there is a chief complaint after the initial assessment (history, physical examination, ECG reading) leading to a standardized work-up or treatment is answered with: no. Further none of the vital signs (body temperature, pulse or heart rate, blood pressure and respiratory rate) are out of range in said patient. Moreover, after initial assessment a working diagnosis cannot be established, especially not with sufficient certainty.

### Non-Specific Complaints: Defining the Endpoint

Due to the broad spectrum of possible diseases underlying a presentation with NSC, a narrow disease-specific endpoint definition is not suitable. In patients with NSC, ED physicians are rather concerned about the task of case identification i.e. to distinguish serious from non-serious outcomes or conditions. Thus, we define a serious condition as any potentially life-threatening condition (e.g. myocardial infarction) or any condition which requires an early intervention to prevent health status deterioration leading to possible morbidity, disability or death (e.g. severe hyponatremia). Obviously, the natural course of an underlying serious condition according to this definition should not be awaited. Moreover, any death occurring within 30-days of the initial ED presentation was judged as a serious condition.

Our definition of a serious condition therefore covers a comprehensive list (Table 1), which was a priori defined and further refined using a modified Delphi technique during three pilot studies where experience on serious conditions accumulated. The association of a NSC and a potential serious condition is particularly likely if a close temporal relationship exists between the development of the NSC and outcome detection.

Threshold levels can be obtained for instance from a Kaplan-Meier analysis, where the occurrence of a disease or the probability of a serious condition and/or death is correlated with the quartiles of the respective markers in the population. According to this analysis, subjects with marker levels above the 75th percentile have a significantly increased risk for getting the diseases according to the invention. This result is further supported by Cox regression analysis with adjustment for classical risk factors. The highest quartile versus all other subjects is highly significantly associated with increased risk for getting a disease or the probability of a serious condition and/or death according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

Other mathematical possibilities to calculate an individual's risk by using the individual's marker level value and other prognostic laboratory and clinical parameters are for instance the NRI (Net Reclassification Index) or the IDI (Integrated Discrimination Index). The indices can be calculated according to Pencina (Pencina M J, et al.: Evaluating the added predictive ability of a new marker: from area under the ROC curve to reclassification and beyond. Stat Med. 2008; 27:157-172).

According to the method, a patient with non-specific complaints is attributed to a risk of getting a serious condition including death when said determined marker peptide level is higher than a predetermined threshold level.

Preferably, the predetermined threshold level of the marker peptide PCT is between 0.02 ng/mL and 0.5 ng/mL, more preferably between 0.02 ng/mL and 0.25 ng/mL, even more preferred between 0.02 ng/mL and 0.1 ng/mL, even more preferred between 0.02 ng/mL and 0.06 ng/mL, most preferred between 0.02 ng/mL and (below) 0.05 ng/mL. In a preferred embodiment the patient with non-specific complaints is attributed to a risk of getting a serious condition when said determined PCT level is higher than 0.1 ng/mL, preferably higher than 0.05 ng/mL, more preferably higher than 0.025 ng/mL.

Preferably, the predetermined threshold level of the marker peptide PRX-4 is between 3 U/mL and 11 U/mL, more preferably between 3 U/mL and 8 U/mL, even more preferred between 3 U/mL and 6 U/mL, even more preferred between 3 U/mL and 5 U/mL, most preferred between 3 U/mL and below 5 U/mL. In a preferred embodiment the patient with non-specific complaints is attributed to a risk of getting a serious condition when said determined PRX-4 level is higher than 11 U/mL, preferably higher than 6 U/mL, more preferably higher than 3 U/mL.

Preferably, the predetermined threshold level of the marker peptide MR-proANP is between 80 pmol/L and 430 pmol/L, more preferably between 80 pmol/L and 330 pmol/L, even more preferred between 80 pmol/L and 185 pmol/L, even more preferred between 80 pmol/L and 140 pmol/L, most preferred between 80 pmol/L and below 140 pmol/L. In a preferred embodiment the patient with non-specific complaints is attributed to a risk of getting a serious condition when said determined MR-proANP level is higher than 430 pmol/L, preferably higher than 185 pmol/L, more preferably higher than 80 pmol/L.

Preferably, the predetermined threshold level of the marker peptide Copeptin is between 5 pmol/L and 80 pmol/L, more preferably between 5 pmol/L and 40 pmol/L, even more preferred between 5 pmol/L and 30 pmol/L, even more preferred between 5 pmol/L and 20 pmol/L, most preferred between 5 pmol/L and 10 pmol/L. In a preferred embodiment the patient with non-specific complaints is attributed to a risk of getting a serious condition when said determined Copeptin level is higher than 80 pmol/L, preferably higher than 30 pmol/L, more preferably higher than 10 pmol/L, even more preferably higher than 5 pmol/L.

Preferably, the predetermined threshold level of the marker peptide MR-proADM is between 0.75 nmol/L and 3 nmol/L, more preferably between 0.75 nmol/L and 2.0 nmol/L, even more preferred between 0.75 nmol/L and 1.5 nmol/L, most preferred between 0.75 nmol/L and 1.0 nmol/L. In a preferred embodiment the patient with non-specific complaints is attributed to a risk of getting a serious condition when said determined MR-proADM level is higher than 3 nmol/L, preferably higher than 2 nmol/L, more preferably higher than 1.5 nmol/L, even more preferably higher than 1.0 nmol/L even more preferably higher than 0.75 nmol/L.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “capture molecules” are molecules which may be used to bind target molecules or molecules of interest, i.e. analytes (e.g. in the context of the present invention the cardiovascular peptide(s)), from a sample. Capture molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, capture molecules may for instance be selected from the group comprising a nucleic acid molecule, a carbohydrate molecule, a PNA molecule, a protein, an antibody, a peptide or a glycoprotein. Preferably, the capture molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

The preferred detection methods comprise immunoassays in various formats such as for instance radioimmunoassay (RIA), chemiluminescence- and fluorescence-immunoassays, Enzyme-linked immunoassays (ELISA), Luminex-based bead arrays, protein microarray assays, and rapid test formats such as for instance immunochromatographic strip tests.

The assays can be homogenous or heterogeneous assays, competitive and non-competitive assays. In a particularly preferred embodiment, the assay is in the form of a sandwich assay, which is a non-competitive immunoassay, wherein the molecule to be detected and/or quantified is bound to a first antibody and to a second antibody. The first antibody may be bound to a solid phase, e.g. a bead, a surface of a well or other container, a chip or a strip, and the second antibody is an antibody which is labeled, e.g. with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem. Biol. 2006 February; 10(1):4-10. PMID: 16376134, incorporated herein by reference).

In a particularly preferred embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

Even more preferred, said labelling system comprises rare earth cryptates or rare earth chelates in combination with a fluorescence dye or chemiluminescence dye, in particular a dye of the cyanine type. In the context of the present invention, fluorescence based assays comprise the use of dyes, which may for instance be selected from the group comprising FAM (5- or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in Kirk-Othmer, Encyclopedia of chemical technology, 4^{th} ed., executive editor, J. I. Kroschwitz; editor, M. Howe-Grant, John Wiley & Sons, 1993, vol. 15, p. 518-562, incorporated herein by reference, including citations on pages 551-562. Preferred chemiluminescent dyes are acridiniumesters.

The term “sample” as used herein refers to a sample of bodily fluid obtained for the purpose of diagnosis, prognosis, or evaluation of a subject of interest, such as a patient. Preferred test samples include blood, serum, plasma, cerebrospinal fluid, urine, saliva, sputum, and pleural effusions. In addition, one of skill in the art would realize that some test samples would be more readily analyzed following a fractionation or purification procedure, for example, separation of whole blood into serum or plasma components.

Thus, in a preferred embodiment of the invention the sample is selected from the group comprising a blood sample, a serum sample, a plasma sample, a cerebrospinal fluid sample, a saliva sample and a urine sample or an extract of any of the aforementioned samples. Preferably, the sample is a blood sample, most preferably a serum sample or a plasma sample.

It is preferred that the plasma- or serum sample has been obtained in a way, by which blood cells potentially containing PRX-4 are quantitatively separated from plasma or serum. This can be achieved for instance by centrifuging the blood sample at least at 3.000 g for at least 20 minutes.

### EXAMPLES

### Basel Non-Specific Complaints Study (BANC Study)

### Study Design

The BANC study is a delayed type cross-sectional diagnostic study. A prospective 30 day to 6 months follow-up was implemented to identify, ascertain and specify underlying conditions that most likely have lead to NSC (Knottnerus 2002. J Clin Epidemiol 55(12):1201-06). The study protocol was approved by the local ethics committee (EKBB 73/07).

### Study Setting and Population

The study took place in the Emergency Department of the University Hospital of Basel, Switzerland. The hospital is a 700-bed primary and tertiary care university hospital and the ED admits over 41,000 patients with a typical ED case-mix per year. All patients admitted to the ED of the University Hospital Basel from May 2007 until November 2007 were consecutively enrolled in order to obtain a sample of the source population of ED self-referred and referred patients with NSC.

### Study Protocol

All adult non-trauma patients with an Emergency Severity Index (ESI) of 2 or 3 (Gilboy et al. 2005. Emergency Severity Version 4: Implementation Handbook. Rockville: Agency for Healthcare Research and Quality) were screened for inclusion (FIG. 1). Patients with an ESI of 4 or 5 were excluded because their resource utilization is low per definition. Furthermore, in ESI 4 and ESI 5 patients, a comprehensive work-up is usually not performed, because a working diagnosis can be generated in most cases. Patients with specific complaints (e.g. chest pain) or a clinical presentation suggestive of a working diagnosis that could be managed using evidence-based management protocols for ED physican use (Laifer and Bingisser 2009. Notfall-Standards, Interdisziplinäre Notfallstation, Universitätsspital Basel. 5. Auflage ISBN 978-3-033-02315-4; EuSEM Task Force. European Curriculum for Emergency Medicine. A document of the EuSEM Task Force on Curriculum approved by the Council of the European Society for Emergency Medicine and by the UEMS Multidisciplinary Joint Committee on Emergency Medicine. 2008. Available at: http://www.eusem.org/downloads/pdfs/Emergency_Medicine_curruculum_final_draft.pdf.), were excluded. In the ease of such an exclusion, physicians were asked to name the “specific complaint” or “working diagnosis” together with the corresponding management protocol (Laifer and Bingisser 2009. Notfall-Standards, Interdisziplinäre Notfallstation, Universitätsspital Basel. 5. Auflage ISBN 978-3-033-02315-4) they applied (FIG. 1). Any patients presenting with recent external laboratory results or specific ECG changes on admission (e.g. STEMI) were not eligible. Similarly, patients with known terminal medical conditions (e.g. end-stage cancer) who were admitted to the ED and were likely to die within the next 30 days were not eligible. Patients were excluded if they were hemodynamically unstable or if the vital parameters were markedly outside the normal range (e.g. systolic blood pressure <90 mmHg, heart rate >120/min, body temperature >38.4 or <35.6 degrees Celsius, respiratory rate >30/min), as management protocols exist for this patient group (ESI 1). In addition, all patients referred from other hospitals were excluded.

ED resident physicians received pre-study training with a lecture and on-site-training on how to correctly apply the BANC protocol. A checklist for the inclusion procedure was openly displayed in our ED. All potentially eligible patients were then prospectively screened for enrolment. Screening started with ESI triage and assessment of vital signs by certified ED triage nurses, history taking, physical examination and electrocardiography reading by ED resident physicians. Laboratory or imaging results were not available at the moment of screening. In order to survey the BANC inclusion procedure, all included patients were reviewed and confirmed by the BANC expert panel physicians.

### Patient Data

The following patient data were obtained as patient data during ED admission and registered on the patient's case report form: demographic baseline data, triage data, complaints, vital signs, Glasgow Coma Scale assessment, medical history, physical examination and electrocardiography reading. Information about activities of daily living according to the Katz index (Katz and Akpom 1976. Med Care 14(5 Suppl):116-8), recent falls or decline of activities, hospitalizations during the previous year, body mass index and weight loss, consumption of alcoholic beverages and cognition (Sunderland et al. 1989. J Am Geriatr Soc 37(8):725-9) were obtained by bedside patient interviews. Several additional variables including a complete list of co-morbidities (Charlson et al. 1987. J Chronic Dis 40(5):373-83) and use of medication were gathered from initial physician reports. Patients received an extensive work-up and were treated at the discretion of the responsible ED physician.

### Patient Follow Up and Outcome Ascertainment

We obtained 30-day and 6-months follow-up data from the patients' primary care physicians or from discharge reports when hospitalization outlasted the 30-day and 6-month period, respectively. Two physicians certified in internal medicine (outcome assessors), blinded to the patients' baseline data reviewed all outcome data. The main outcome of interest was the occurrence of a serious condition during the 30-day follow-up. In addition, we sub-classified the group: An “acute new condition” was defined as a newly diagnosed disease (e.g. pneumonia). A “deterioration of a chronic condition” was defined as the deterioration of a pre-existing disease ultimately leading to further medication or other intervention (e.g. worsening of chronic heart failure). An “acute event in a chronic condition” was defined as an acute unexpected incident or complication in a pre-existing condition (e.g. pulmonary embolism in a patient with known cancer). If symptoms were suggestive to be iatrogenic or caused by well-known medication side-effects, we sub-classified them as “iatrogenic or therapy-induced”, irrespective whether treatment was initiated or discontinued by either the physician or the patient. Finally, if no somatic disease explained the patient's NSC after discharge and complete follow-up, the classification non-organic or functional was chosen.

In regular periods, the outcome assessors reviewed all patient records in order to establish a final diagnosis according to the 10th international classification of diseases and related health problems (ICD-10). According to these guidelines, “main condition” was chosen as the one which was most closely related to the patient's initial presentation, receiving the highest amount of resources for treatment. If no diagnosis could be made, the main symptom, or abnormal finding was chosen to be “main condition”, using a descriptive diagnosis, such as described in chapter R of ICD-10.

In case the judgement of the two outcome assessors disagreed, the patients' records were reviewed and consensus sought in the BANC expert panel. The expert panel consisted of two board-certified physicians with at least ten years of experience in the field of internal medicine.

### Basel Non-Specific Complaints Study III (BANC Study III)

### Study Design

The BANC study is a delayed type cross-sectional diagnostic study. A prospective 30 day follow-up was implemented. The study protocol was approved by the local ethics committee.

### Study Setting and Population

The study took place in the Emergency Department of the University Hospital of Basel, Switzerland (see BANC Study). All patients admitted to the ED of the University Hospital Basel from April 2009 until December 2010 were consecutively enrolled in order to obtain a sample of the source population of ED self-referred and referred patients with NSC.

### Study Protocol/Patient Data/Patient Follow Up and Outcome Ascertainment

The study protocol, outcome ascertainment, patient data obtained and the patient follow up (for 30 days) were the same as described in the respective sections for the BANC study.

### Marker Measurements

### MR-proANP

MR-proANP was detected using novel fully automated sandwich immunoassay systems on the B.R.A.H.M.S KRYPTOR (B.R.A.H.M.S GmbH, Hennigsdorf/Berlin, Germany) This random access analyzer employs the sensitive Time Resolved Amplified Cryptate Emission (TRACE) technology, based on a non-radioactive-transfer between 2 fluorophores, europium cryptate and XL665. The automated assay is based essentially on the sandwich chemiluminescence assay which is described in detail elsewhere (Morgenthaler et al. 2004. Clin Chem 50:234-6), and which was used in other studies (Khan et al. 2008. J Am Coll Cardiol 51:1857-64; Gegenhuber et al. 2006. Clin Chem 52: 827-31).

For MR-proANP detection, 14 μl of patients serum were incubated for 14 min. The measuring range was 0-10000 pmol/L, the limit of detection 2.1 pmol/L, and the limit of quantitation 4.5 pmol/L. The intra assay CV was 1.2% and the inter laboratory CV 5.4%. This assay uses the same antibody pair as the reference assay (Morgenthaler et al. 2004. Clin Chem 50: 234-6), and the correlation between the two assay systems was r=0.99.

### MR-proADM

MR-proADM is detected using a novel fully automated sandwich immunoassay system on the B.R.A.H.M.S KRYPTOR (B.R.A.H.M.S GmbH, Hennigsdorf/Berlin, Germany) (Caruhel et al. 2009. Clin Biochem 42:725-8). This random access analyzer employs the sensitive Time Resolved Amplified Cryptate Emission (TRACE) technology, based on a non-radioactive-transfer between 2 fluorophores, europium cryptate and XL665. This automated assay is based essentially on the sandwich chemiluminescence assay which is described in detail elsewhere (Morgenthaler et al. 2005 Clin Chem 51:1823-9), and which was used in other studies (Khan et al. 2007. J Am Coll Cardiol 49: 1525-32; Gegenhuber et al. 2007. J Card Fail 13:42-9).

For MR-proADM detection, 26 μl serum is incubated for 29 min. The measuring range was 0-100 mmol/L, the limit of detection and limit of quantification were 0.05 and 0.23 nmol/L, respectively. The intra assay CV is 1.9% and the inter laboratory CV is 9.8%. This assay uses the same antibody pair as described in detail elsewhere (Morgenthaler et al. 2005. Clin Chem 51: 1823-9), and the correlation between the two assay systems is r=0.99.

### CT-proET-1

CT-proET-1 levels can be measured with a chemiluminescence sandwich immunoassay with a lower detection limit of 0.4 pmol/L (Papassotiriou et al. 2006. Clin Chem 52: 1144-51). In 326 healthy individuals (150 male and 176 female) CT-proET-1 values followed a Gaussian distribution with a mean (SD) of 44.3 (10.6) pmol/L and a range of 10.5-77.4 pmol/L. Mean CT-proET-1 concentrations in males and females are not significantly different but significantly correlated with age. The intra assay imprecision (CV) is <5% and the inter laboratory CV was <10%.

### Copeptin

CT-proAVP (Copeptin) levels were measured with a chemiluminescence sandwich immunoassay with a lower detection limit of 1.7 pmol/L (Morgenthaler et al. 2006. Clin Chem 52:112-9). In 359 healthy individuals (153 men and 206 women) median CT-proAVP levels were 4.2 pmol/L ranging from 1.0-13.8 pmol/L. Median concentrations of CT-proAVP differed significantly between male and female. There was no correlation between CT-proAVP levels and age. The inter laboratory CV was <20% and the intra assay CV was <10% for samples >2.25 pmol/L.

### Peroxiredoxin-4

Peroxiredoxin-4 (PRX-4) was measured using a newly developed chemiluminescence sandwich immunoassay as described recently (Schulte et al. 2010. Clin Chim Acta 411:1258-1263). The functional assay sensitivity (interassay CV <20%) was 0.51 arb. U/L. In 272 healthy blood donors (44% men) median PRX-4 levels were 0.71 arb.U/L ranging from 0.15-5.1 arb.U/L. There was a weak significant difference between male and female and no correlation between Prx-4 levels and age.

### Procalcitonin

PCT was measured using an ultrasensitive commercially available test system with a functional assay sensitivity of 0.007 ng/mL as described in Morgenthaler et al. (Morgenthaler et al. 2002. Clin Chem 48:788-790).

### Data Analysis

Descriptive analyses were performed to summarize the baseline characteristics of the study population and to describe disease manifestations underlying serious or non-serious conditions. Descriptive statistics given for continuous variables are median (range), for categorical variables we report n (percent). Box-and-whisker plots of single marker values were used to summarize the distribution of marker values in specific subgroups. For prediction of death within 30 days or 6 months Cox regression models were used. To illustrate the ability of the different markers for mortality prediction, we calculated Kaplan-Meier survival curves and stratified patients by marker tertiles. In addition, time-dependent receiver operating characteristics (ROC) plots were performed. A receiver operating characteristic is a graphical plot of the sensitivity vs. (1—specificity), for the binary outcome (death, serious condition, etc.) as its cut off is varied. For all other outcomes, logistic regression models were performed, as well as ROC analysis and plots. Sensitivity (the proportion of actual positives which are correctly identified as such by a biomarker) and specificity (proportion of negatives which are correctly identified) were calculated for selected cut-offs.

### Results

### Study Population (BANC Study):

A total of 438 patients were included into the study. 28 (6.4%) and 53 (12.1%) of these patients died within 30 days and 6 months, respectively. 256 patients had a serious outcome within 30 days as defined in table 1 (58.4%). 177 of patients were hospitalized >30 days (40.4%) and 188 of all patients were admitted to the Intensive Care Unit (ICU)>10 days (42.9%).

Baseline characteristics of the study population are presented in Table 2. Median age was 80 years (range 22-101), 85.6% of subjects were older than 64 years. Almost two third (65%) of the study population was female. Study patients had a median of 5 comorbidities and took 5 different medicaments daily. The median Charlson Comorbidity Index was 2 (Charlson et al. 1987. J Chronic Dis. 40: 373-383) and 43.4% of the study population was dependent in at least one activity of daily living (ADL) (Katz et al. 1970. Gerontologist 10:20-30). The majority (97.7%) of the patients were classified to ESI 3 and therefore needed more than one external resource in the ED.

Box-and-whisker plots of single marker values for the prediction of death within 30 days are shown in FIGS. 2 to 5. MR-proANP, Copeptin, PCT and PRX-4 concentrations were significantly higher in non-survivors than in survivors, respectively (Kruskal-Wallis test, p <0.001 for all four marker peptides).

Receiver operating characteristics for the single markers are shown in FIGS. 6 to 9 (for the prediction of death within 30 days), FIGS. 14 to 17 (for the prediction of serious outcome including death within 30 days) and FIGS. 18 to 21 (for the prediction of death within 6 months). Different cut-off values were used to determine the corresponding sensitivity and specificity for each marker to predict death within 30 days and 6 months, respectively, as well as to predict serious outcome within 30 days (Tables 8 to 11).

To illustrate the prognostic value of the marker peptides, Kaplan-Meier survival curves were calculated for each single marker, dividing the patients into tertiles depending on the respective marker concentrations. The Kaplan-Meier survival curves are shown in FIGS. 10 to 13 (for death within 30 days) and FIGS. 22 to 25 (for death within 6 months), respectively. As shown in FIGS. 10 to 13, higher mortality rates within 30 days were observed, when MR-proANP, Copeptin, PCT and PRX-4 concentrations, respectively, at ED presentation were in the third tertile compared to the first and second. Similarly, as shown in FIGS. 22 to 25, higher mortality rates within 6 months were observed, when MR-proANP, Copeptin, PCT and PRX-4 concentrations, respectively, at ED presentation were in the third tertile compared to the first and second.

The overall prognostic accuracy of the marker peptides was assessed using uni- and multivariate Cox regression analyses (Tables 3 to 7). In univariate models, each marker peptide was used a) to predict death within 30 days after presentation (Table 3), b) to predict the occurrence of a serious condition within 30 days after presentation (Table 4), c) to predict the admission to ICU (with a stay on ICU of at least 10 days) within 30 days after presentation (Table 5), d) to predict hospitalization of at least 30 days after presentation (Table 6) and to predict death within 6 months after presentation (Table 7).

For example, to predict death within 30 days after presentation of the patient to the ED, PRX-4 (C-index=0.749) showed better prediction than Copeptin (C-index=0.724) (Table 3). The bivariable model including PRX-4 and Copeptin (C-index=0.783) allows a significantly better prediction than the univariate PRX-4 model (p <0.001) and the Copeptin model (p <0.001). A model including PRX-4 and Copeptin in addition to age and gender was significantly better than the model using the two marker peptides only (added χ² of 4.65). Moreover, adding the two marker peptides PRX-4 and Copeptin to the univariate model of Katz ADL or Charlson Comorbidity Index (CCI) gave significantly better results than using the two marker peptides alone or in combination, with a C-index of 0.788 for the model combining PRX-4, Copeptin and CCI, and a C-index of 0.807 for the model combining PRX-4, Copeptin and Katz ADL (Table 3).

### Study Population (BANC Study III):

A total of 504 patients were included into the study. The median age was 82 years and 196 patients (38.9%) were male. 33 (6.5%) of these patients died within 30 days. 276 patients had a serious outcome within 30 days as defined in table 1 (54.8%). 203 of all patients were admitted to the Intensive Care Unit (ICU) ≧10 days (40.3%).

Baseline characteristics of the study population are presented in Table 12. Median age was 82 years (range 75-87), 88.9% of subjects were older than 64 years. Almost two third (61.1%) of the study population was female. The median Charlson Comorbidity Index was 2 (Charlson et al. 1987. J Chronic Dis. 40: 373-383) and 54.5% of the study population was dependent in at least one activity of daily living (ADL) (Katz et al. 1970. Gerontologist 10:20-30).

Box-and-whisker plots of single marker values for the prediction of death within 30 days are shown in FIGS. 26 to 30. MR-proANP, Copeptin, PCT, PRX-4 and MR-proADM concentrations were significantly higher in non-survivors than in survivors, respectively (Kruskal-Wallis test, p <0.001 for all five marker peptides).

Receiver operating characteristics for the single markers are shown in FIGS. 31 to 35 for the prediction of death within 30 days, and in FIGS. 41 to 45 for the prediction of serious outcome including death within 30 days. Different cut-off values were used to determine the corresponding sensitivity and specificity for MR-proADM to predict death within 30 days as well as to predict serious outcome within 30 days (Table 13). The cut-off values for the determination of corresponding sensitivity and specificity to predict death and serious outcome, respectively, within 30 days for MR-proANP, Copeptin, PCT and PRX-4 were similar to the values obtained in the BANC study (data not shown).

To illustrate the prognostic value of the marker peptides, Kaplan-Meier survival curves were calculated for each single marker, dividing the patients into tertiles depending on the respective marker concentrations. The Kaplan-Meier survival curves are shown in FIGS. 36 to 40 (for death within 30 days). As shown in FIGS. 36 to 40, higher mortality rates within 30 days were observed, when MR-proANP, Copeptin, PCT, PRX-4 and MR-proADM concentrations, respectively, at ED presentation were in the third tertile compared to the first and second.

The overall prognostic accuracy of the marker peptides was assessed using uni- and multivariate Cox regression analyses (Tables 14, 15 and 16). In univariate models, each marker peptide was used a) to predict death within 30 days after presentation (Table 14), b) to predict the admission to ICU (with a stay on ICU of at least 10 days) within 30 days after presentation (Table 15) and c) to predict the occurrence of a serious condition within 30 days after presentation (Table 16).

For example, to predict death within 30 days after presentation of the patient to the ED, PRX-4 (C-index=0.719) showed similar prediction compared to Copeptin (C-index=0.723) (Table 14). The bivariable model including PRX-4 and Copeptin (C-index=0.76) allows a significantly better prediction than the univariate PRX-4 model (p <0.001) and the Copeptin model (p <0.001). Adding the two marker peptides PRX-4 and Copeptin to the univariate model of Katz ADL gave a significantly better result than using the two marker peptides alone or in combination, with a C-index of 0.819.

### FIGURE DESCRIPTION

FIG. 1: Identification of patients with non-specific complaints in the BANC study and BANC study III

FIG. 2: Box-and-whisker plot of MR-proANP values for the prediction of death in patients with NSC within 30 days (BANC study)

FIG. 3: Box-and-whisker plot of Copeptin values for the prediction of death in patients with NSC within 30 days (BANC study)

FIG. 4: Box-and-whisker plot of PCT values for the prediction of death in patients with NSC within 30 days (BANC study)

FIG. 5: Box-and-whisker plot of PRX-4 values for the prediction of death in patients with NSC within 30 days (BANC study)

FIG. 6: ROC plot for MR-proANP for the prediction of death in patients with NSC within 30 days (AUC=0.67) (BANC study)

FIG. 7: ROC plot for Copeptin for the prediction of death in patients with NSC within 30 days (AUC=0.71) (BANC study)

FIG. 8: ROC plot for PCT for the prediction of death in patients with NSC within 30 days (AUC=0.70) (BANC study)

FIG. 9: ROC plot for PRX-4 for the prediction of death in patients with NSC within 30 days (AUC=0.73) (BANC study)

FIG. 10: Kaplan-Meier survival curves (death within 30 days) by tertiles of MR-proANP for patients with NSC (BANC study)

FIG. 11: Kaplan-Meier survival curves (death within 30 days) by tertiles of Copeptin for patients with NSC (BANC study)

FIG. 12: Kaplan-Meier survival curves (death within 30 days) by tertiles of PCT for patients with NSC (BANC study)

FIG. 13: Kaplan-Meier survival curves (death within 30 days) by tertiles of PRX-4 for patients with NSC (BANC study)

FIG. 14: ROC plot for MR-proANP for the prediction of serious outcome in patients with NSC within 30 days (AUC=0.61) (BANC study)

FIG. 15: ROC plot for Copeptin for the prediction of serious outcome in patients with NSC within 30 days (AUC=0.64) (BANC study)

FIG. 16: ROC plot for PCT for the prediction of serious outcome in patients with NSC within 30 days (AUC=0.67) (BANC study)

FIG. 17: ROC plot for PRX-4 for the prediction of serious outcome in patients with NSC within 30 days (AUC=0.59) (BANC study)

FIG. 18: ROC plot for MR-proANP for the prediction of death in patients with NSC within 6 months (AUC=0.59) (BANC study)

FIG. 19: ROC plot for Copeptin for the prediction of death in patients with NSC within 6 months (AUC=0.66) (BANC study)

FIG. 20: ROC plot for PCT for the prediction of death in patients with NSC within 6 months (AUC=0.62) (BANC study)

FIG. 21: ROC plot for PRX-4 for the prediction of death in patients with NSC within 6 months (AUC=0.68) (BANC study)

FIG. 22: Kaplan-Meier survival curves (death within 6 months) by tertiles of MR-proANP for patients with NSC (BANC study)

FIG. 23: Kaplan-Meier survival curves (death within 6 months) by tertiles of Copeptin for patients with NSC (BANC study)

FIG. 24: Kaplan-Meier survival curves (death within 6 months) by tertiles of PCT for patients with NSC (BANC study)

FIG. 25: Kaplan-Meier survival curves (death within 6 months) by tertiles of PRX-4 for patients with NSC (BANC study)

FIG. 26: Box-and-whisker plot of MR-proANP values for the prediction of death in patients with NSC within 30 days (BANC study III)

FIG. 27: Box-and-whisker plot of Copeptin values for the prediction of death in patients with NSC within 30 days (BANC study III)

FIG. 28: Box-and-whisker plot of PCT values for the prediction of death in patients with NSC within 30 days (BANC study III)

FIG. 29: Box-and-whisker plot of PRX-4 values for the prediction of death in patients with NSC within 30 days (BANC study III)

FIG. 30: Box-and-whisker plot of MR-proADM values for the prediction of death in patients with NSC within 30 days (BANC study III)

FIG. 31: ROC plot for MR-proANP for the prediction of death in patients with NSC within 30 days (AUC=0.697) (BANC study III)

FIG. 32: ROC plot for Copeptin for the prediction of death in patients with NSC within 30 days (AUC=0.723) (BANC study III)

FIG. 33: ROC plot for PCT for the prediction of death in patients with NSC within 30 days (AUC=0.69) (BANC study III)

FIG. 34: ROC plot for PRX-4 for the prediction of death in patients with NSC within 30 days (AUC=0.719) (BANC study III)

FIG. 35: ROC plot for MR-proADM for the prediction of death in patients with NSC within 30 days (AUC=0.732) (BANC study III)

FIG. 36: Kaplan-Meier survival curves (death within 30 days) by tertiles of MR-proANP for patients with NSC (BANC study III)

FIG. 37: Kaplan-Meier survival curves (death within 30 days) by tertiles of Copeptin for patients with NSC (BANC study III)

FIG. 38: Kaplan-Meier survival curves (death within 30 days) by tertiles of PCT for patients with NSC (BANC study III)

FIG. 39: Kaplan-Meier survival curves (death within 30 days) by tertiles of PRX-4 for patients with NSC (BANC study III)

FIG. 40: Kaplan-Meier survival curves (death within 30 days) by tertiles of MR-proADM for patients with NSC (BANC study III)

FIG. 41: ROC plot for MR-proANP for the prediction of serious outcome in patients with NSC within 30 days (AUC=0.706) (BANC study III)

FIG. 42: ROC plot for Copeptin for the prediction of serious outcome in patients with NSC within 30 days (AUC=0.722) (BANC study III)

FIG. 43: ROC plot for PCT for the prediction of serious outcome in patients with NSC within 30 days (AUC=0.73) (BANC study III)

FIG. 44: ROC plot for PRX-4 for the prediction of serious outcome in patients with NSC within 30 days (AUC=0.694) (BANC study III)

FIG. 45: ROC plot for MR-proADM for the prediction of serious outcome in patients with NSC within 30 days (AUC=0.732) (BANC study III)

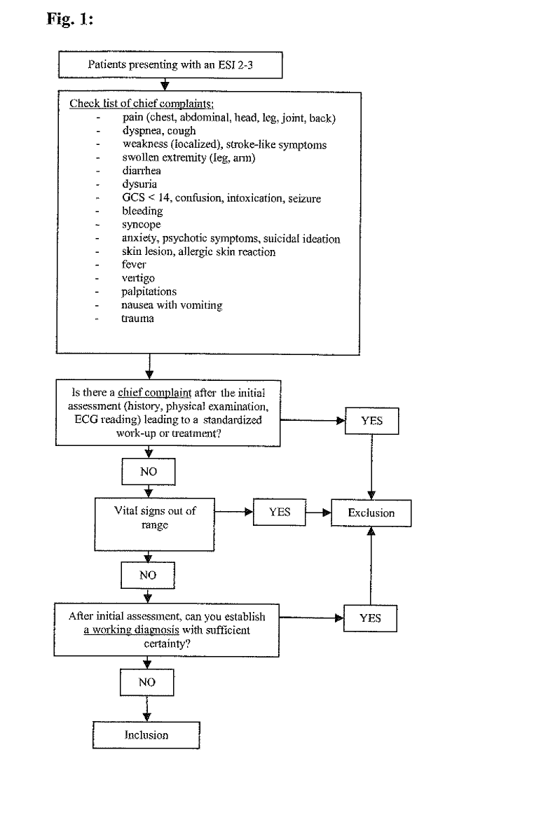

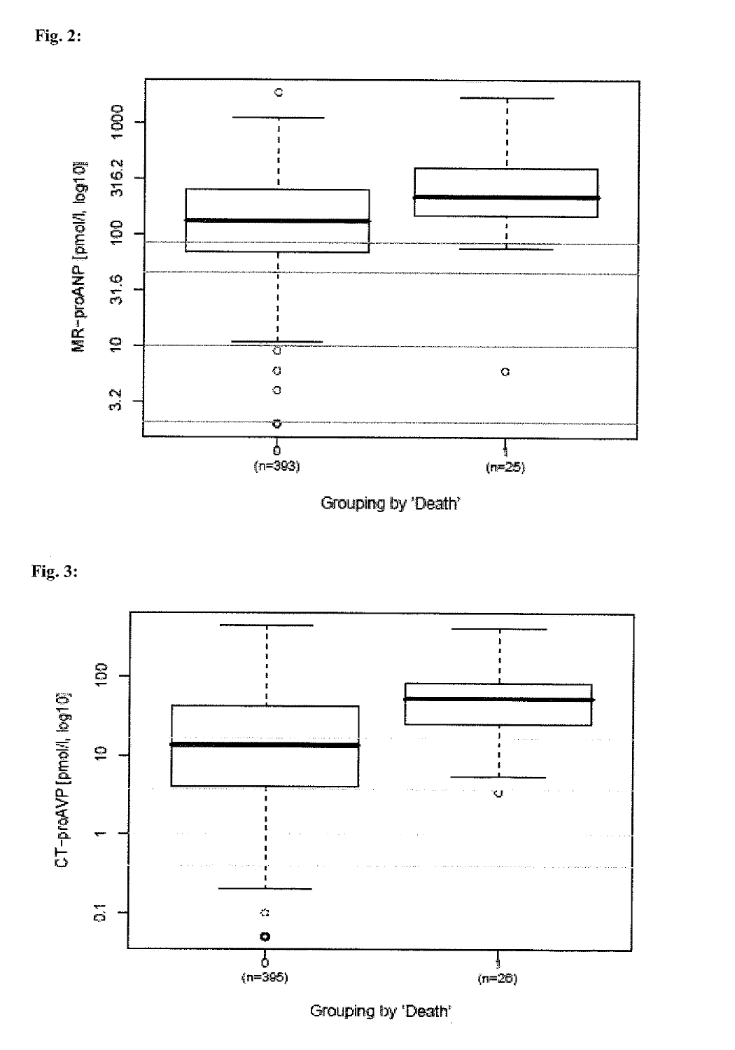

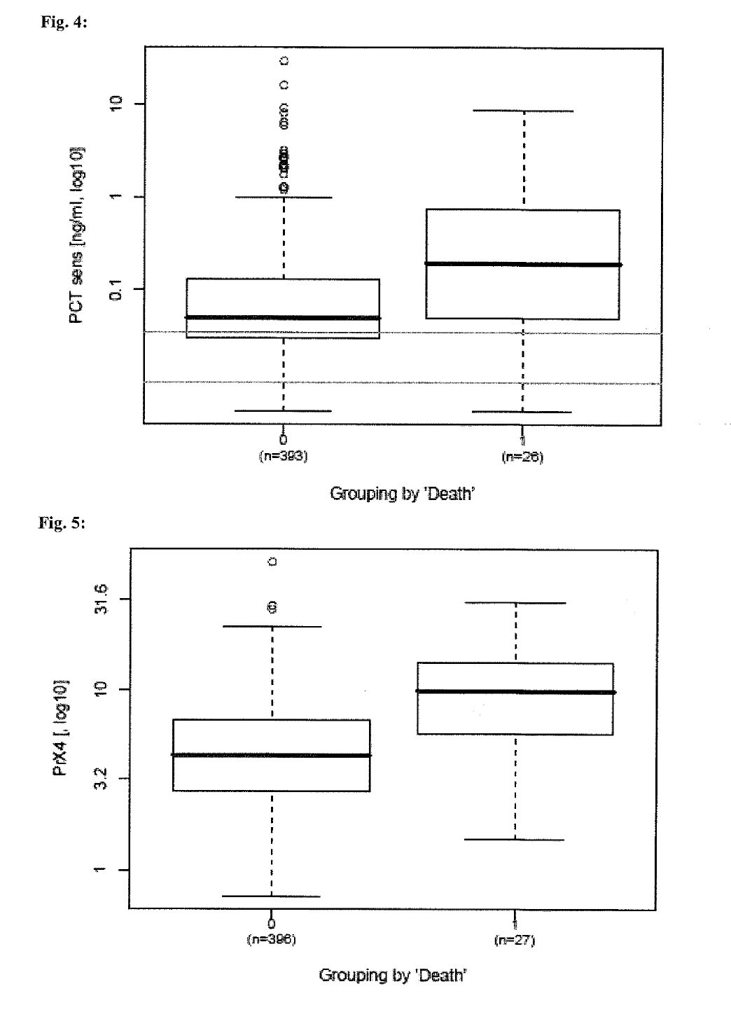

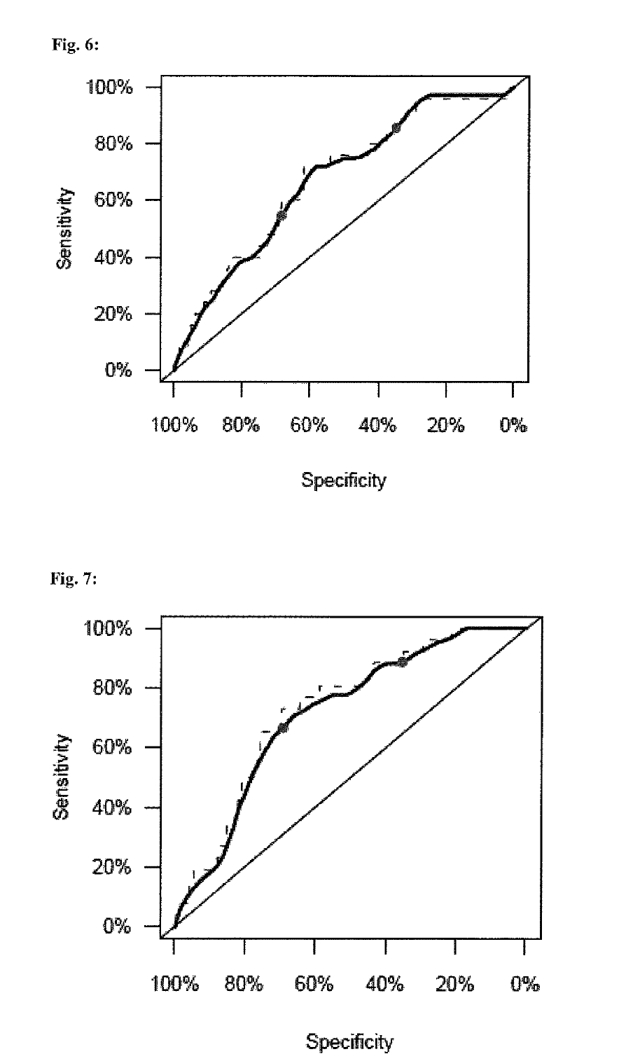

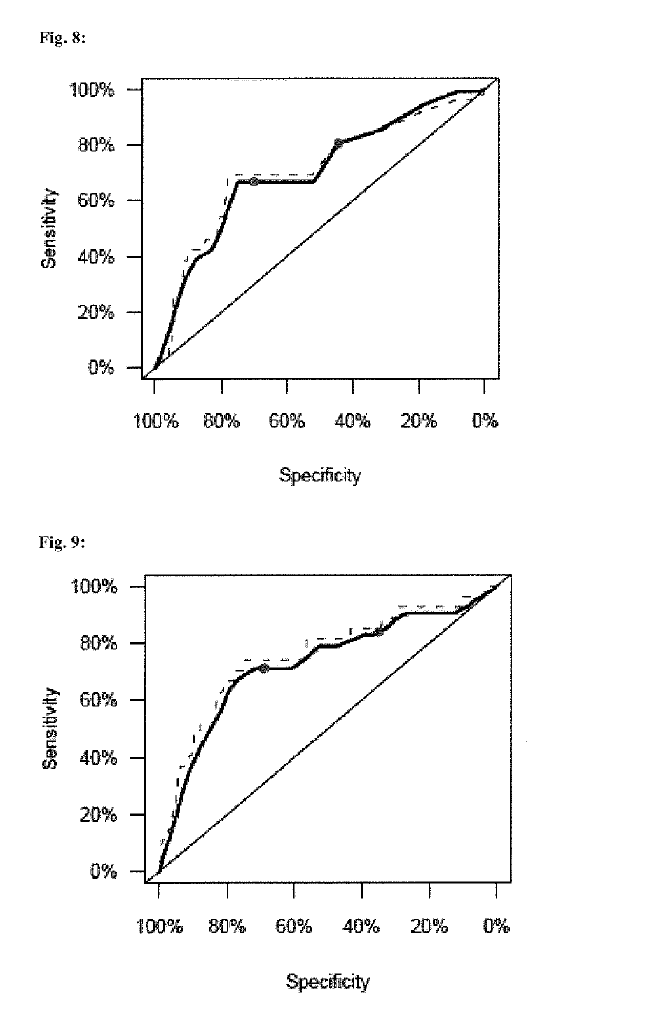

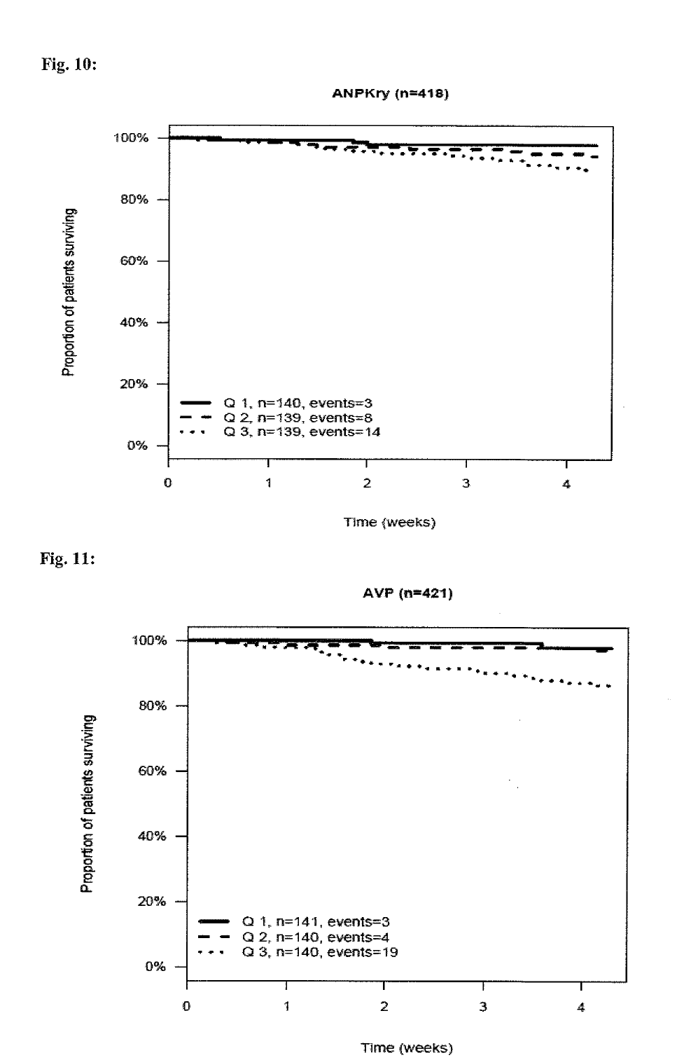

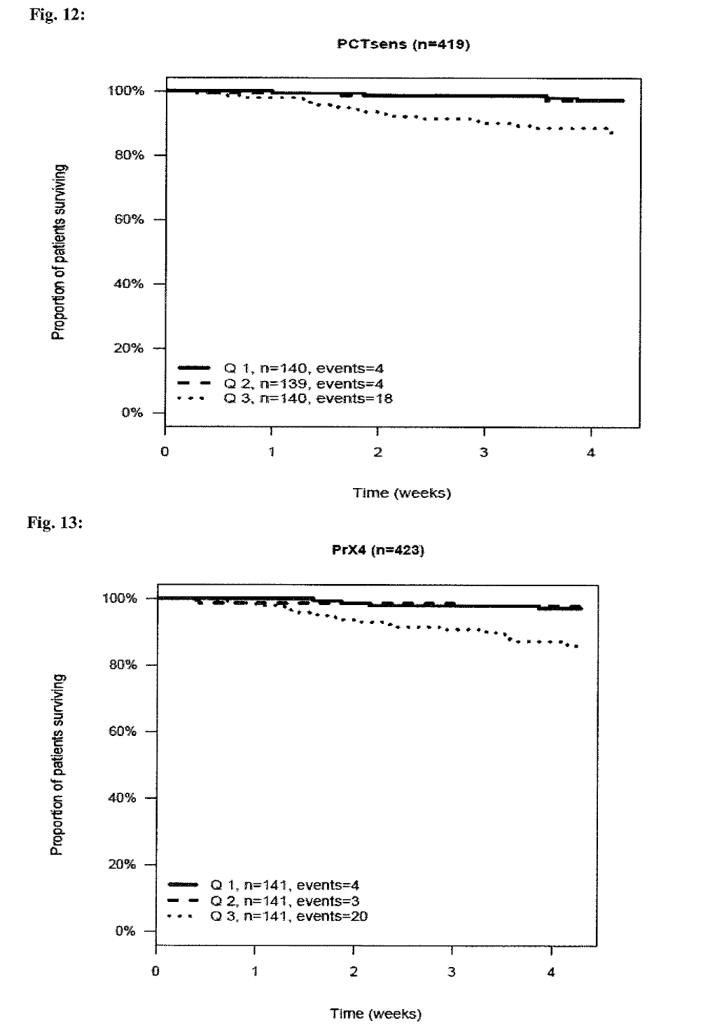

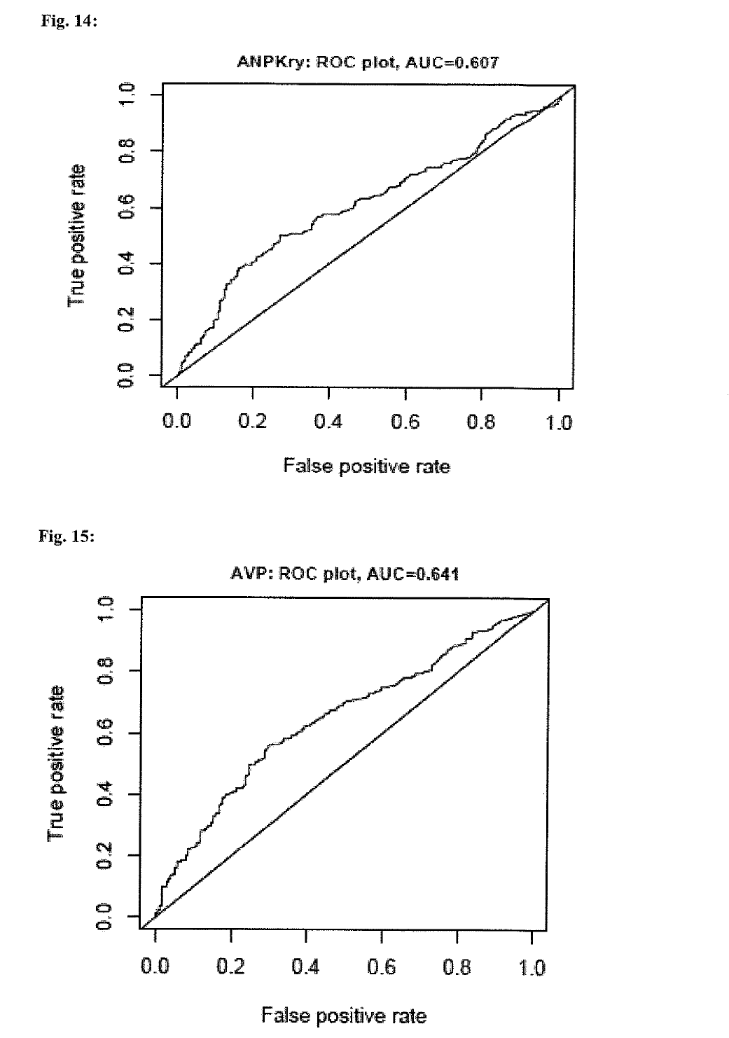

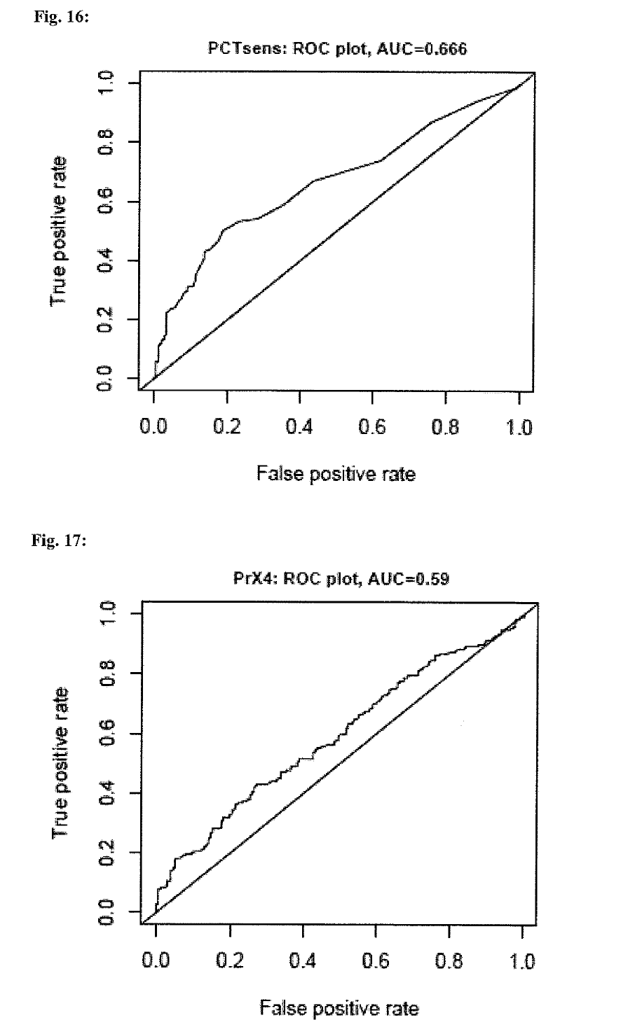

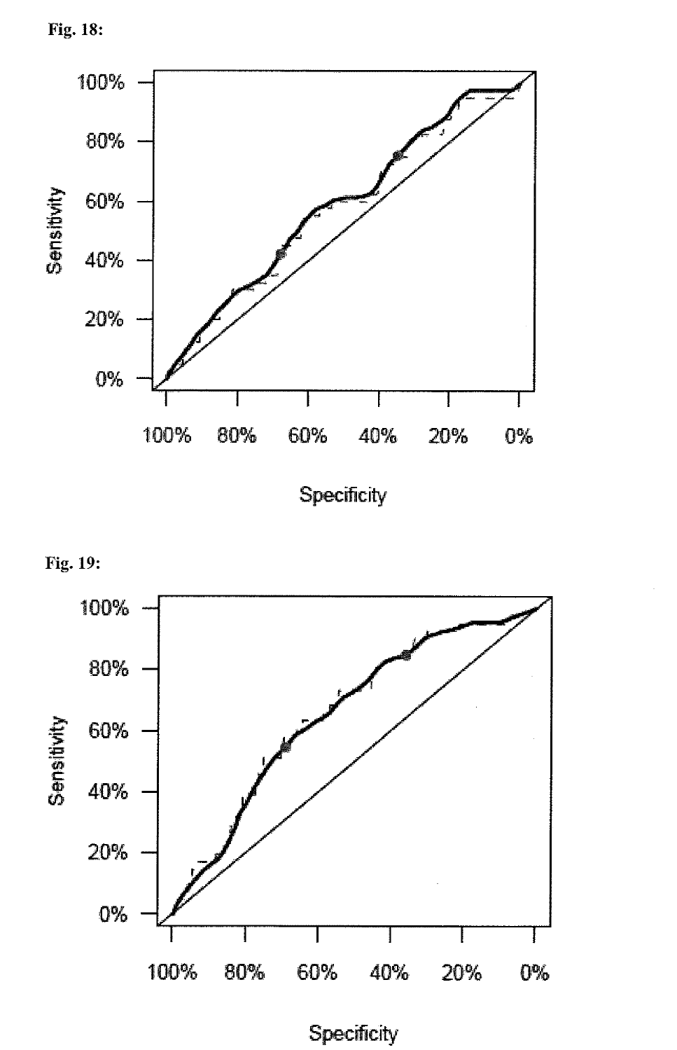

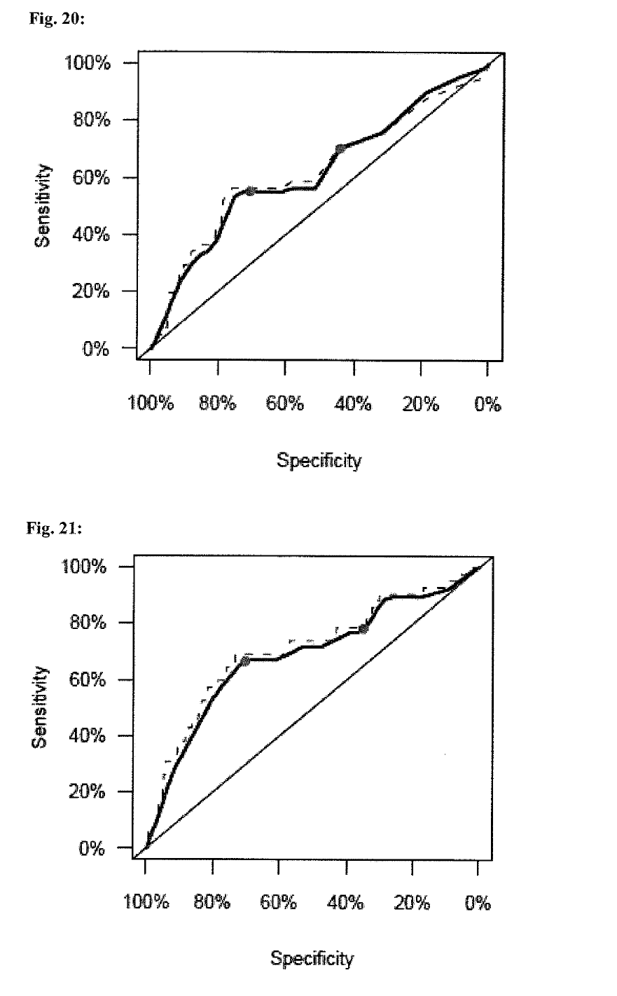

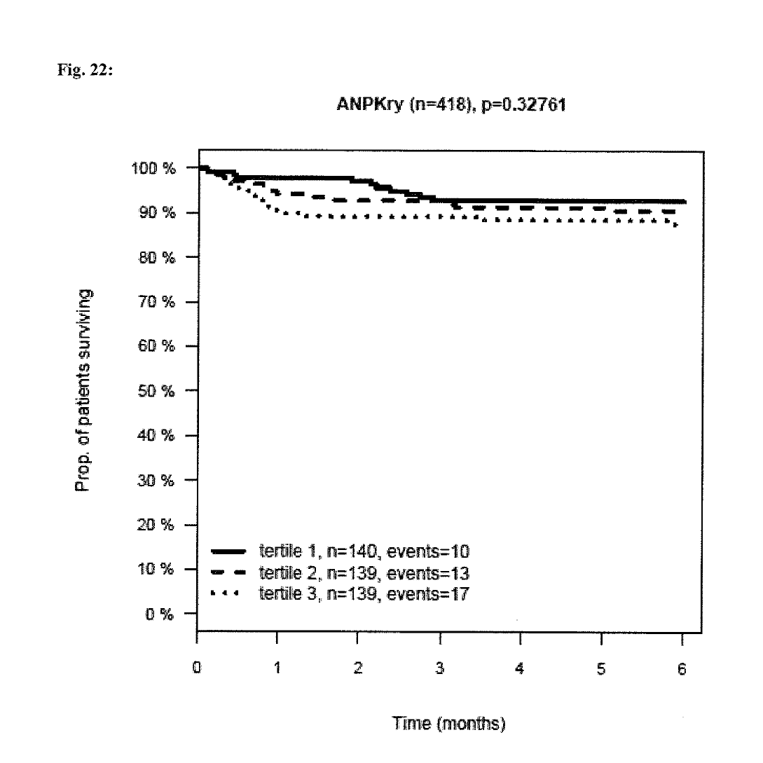

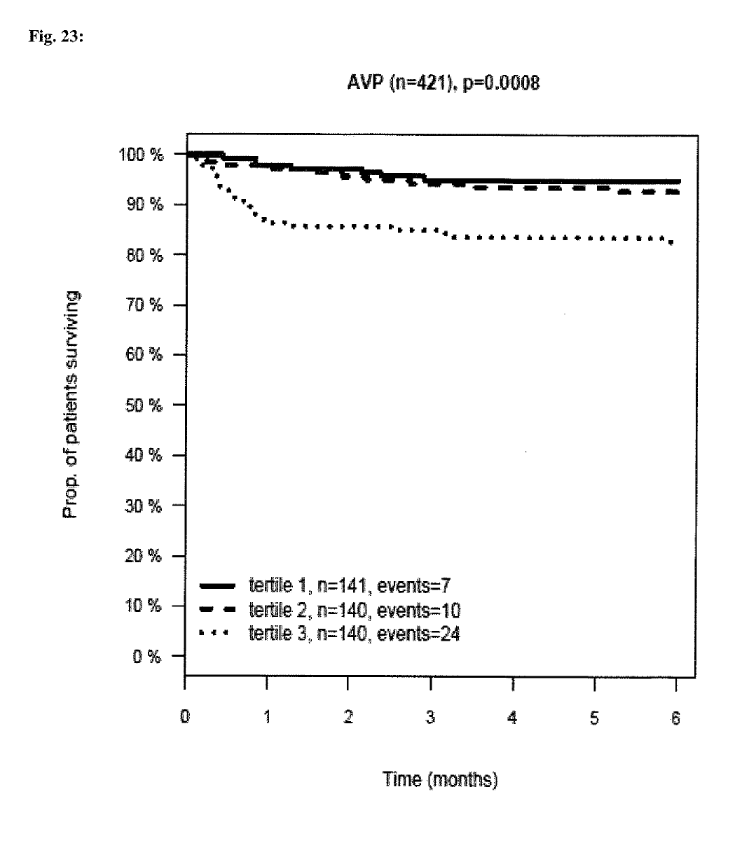

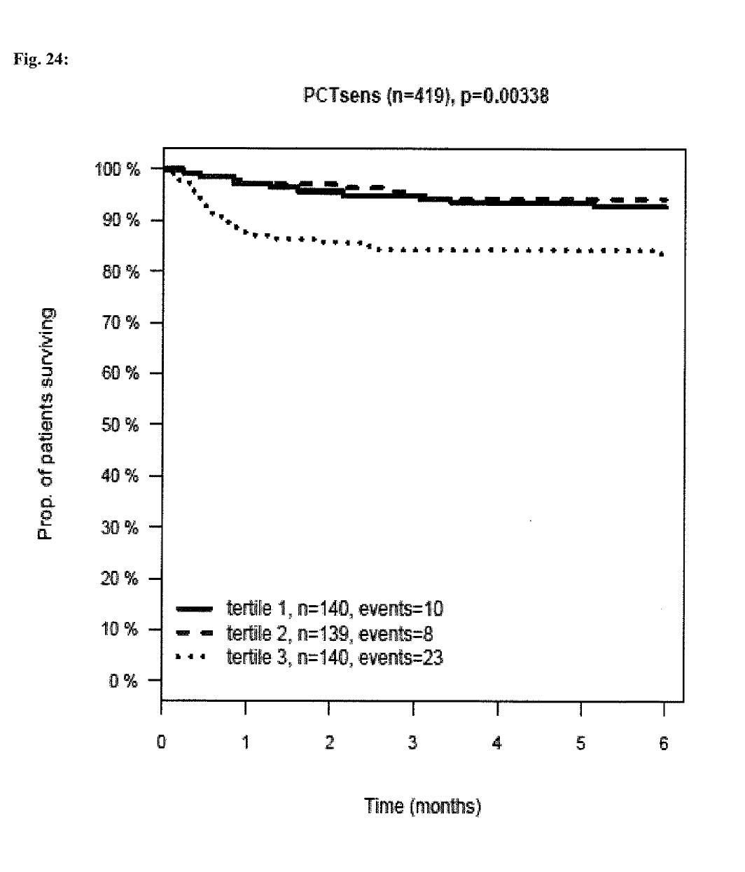

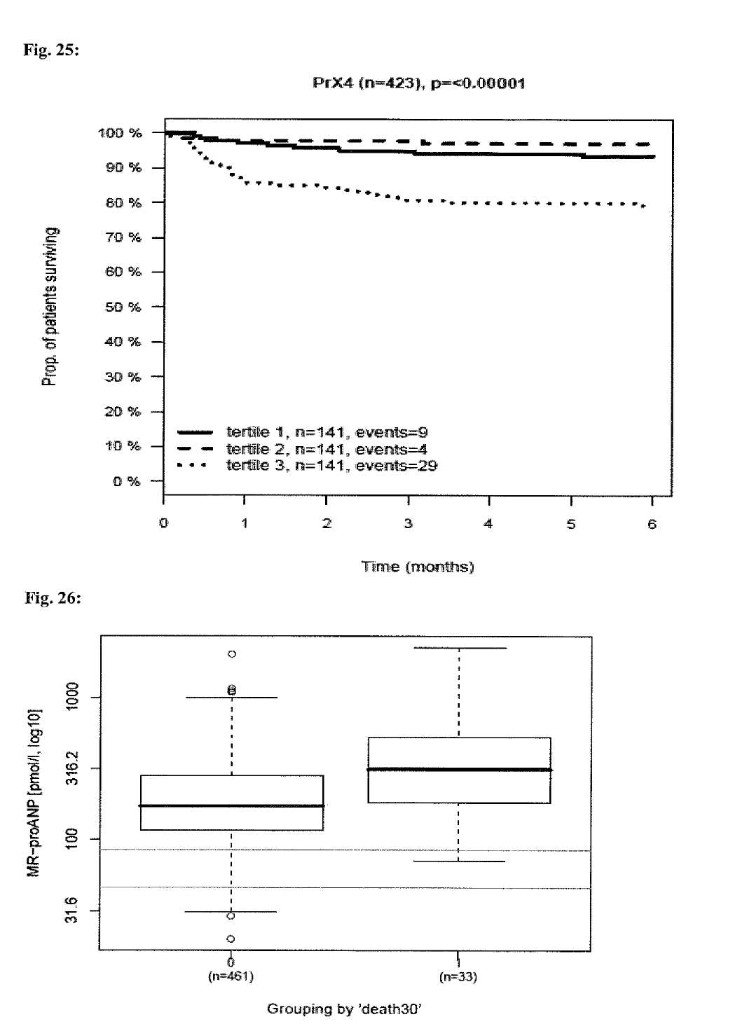

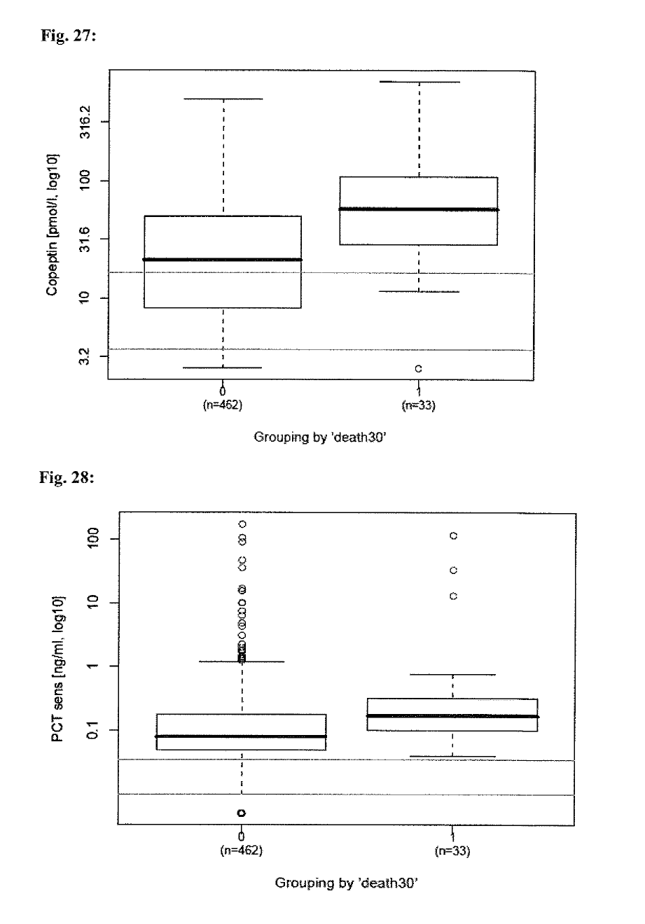

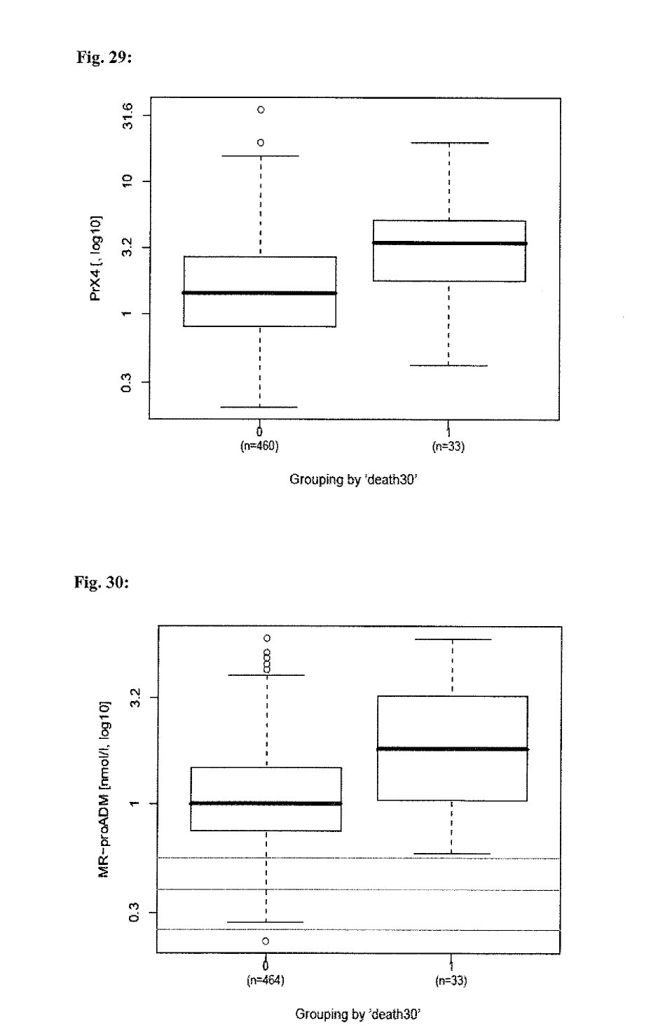

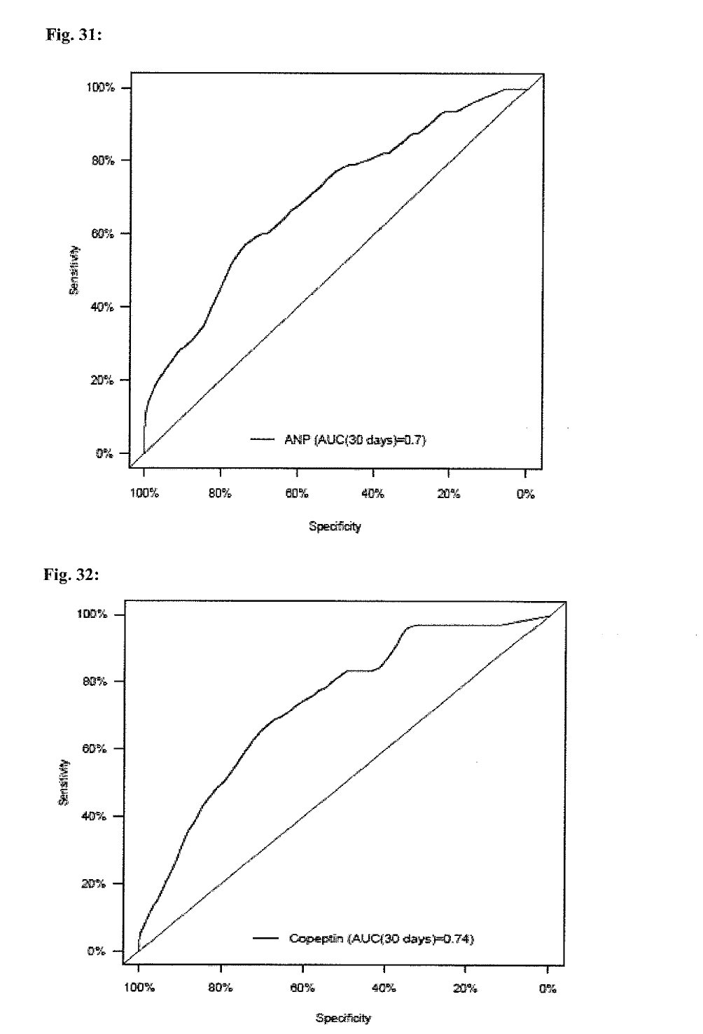

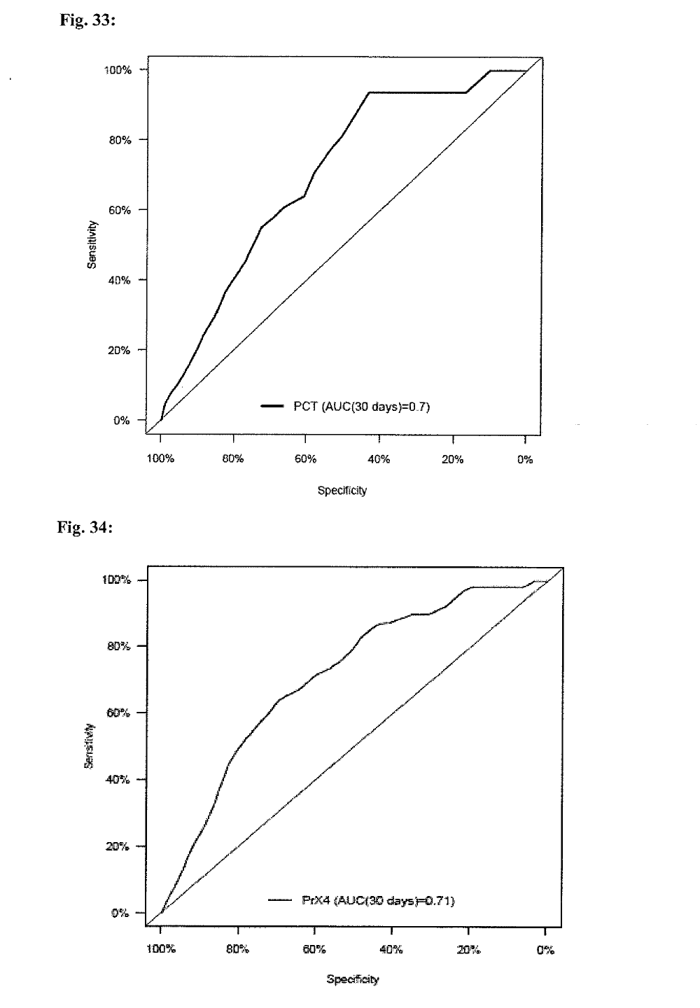

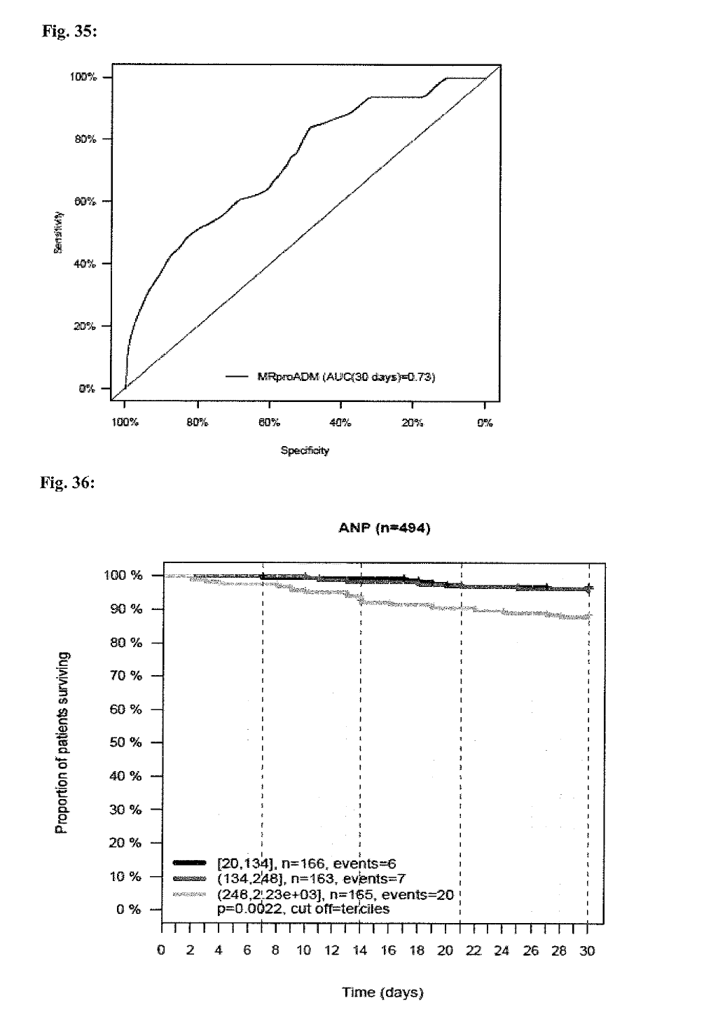

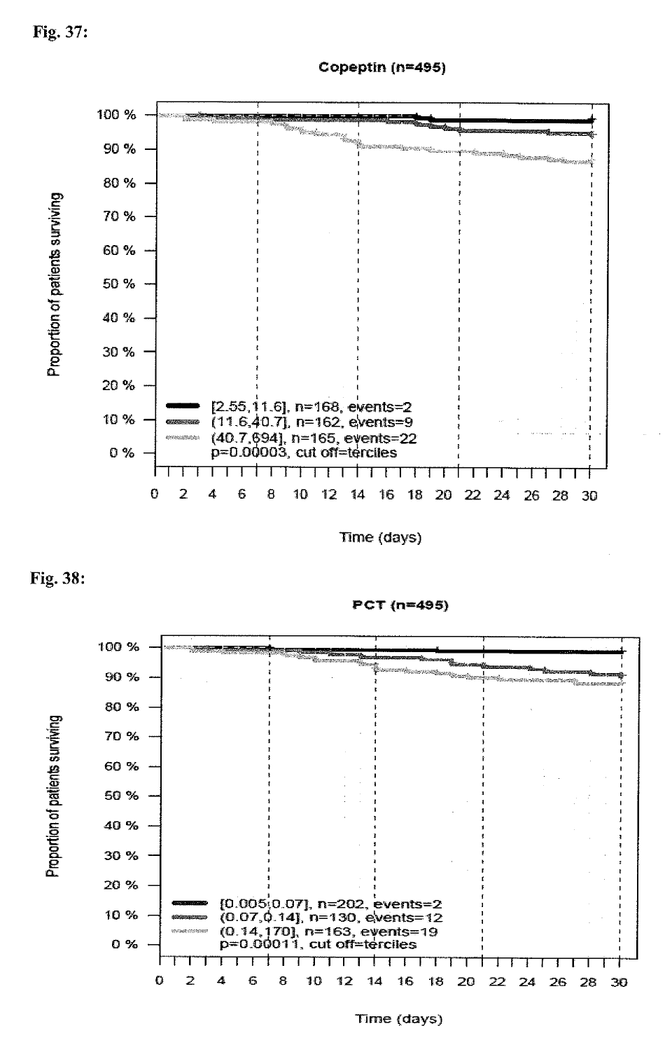

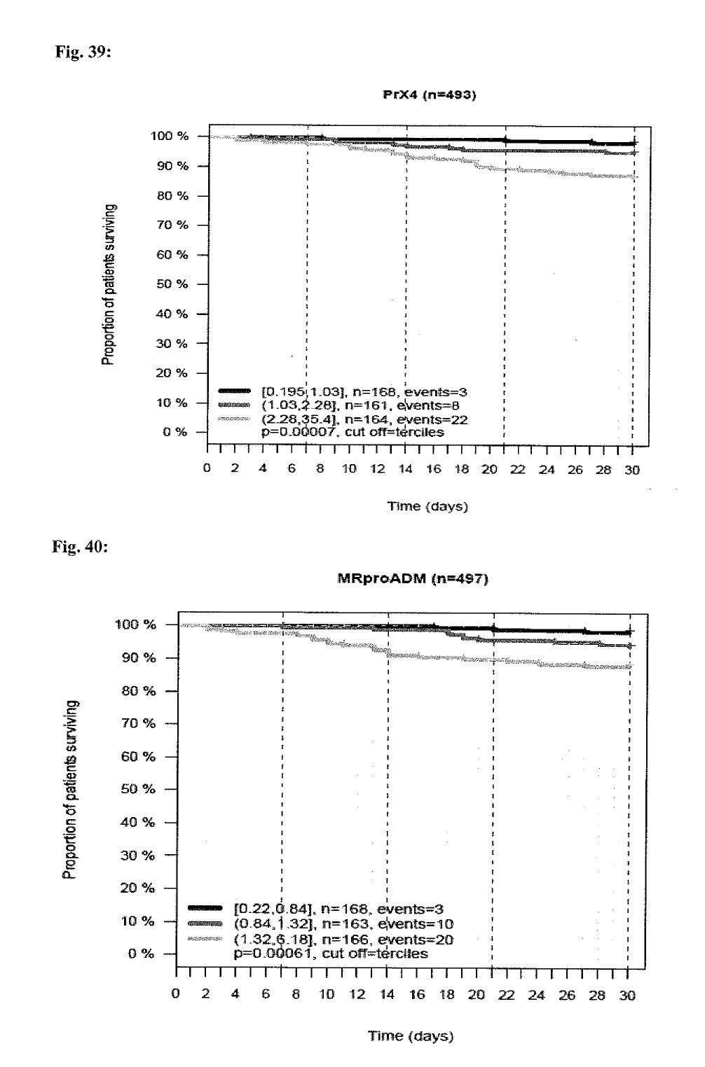

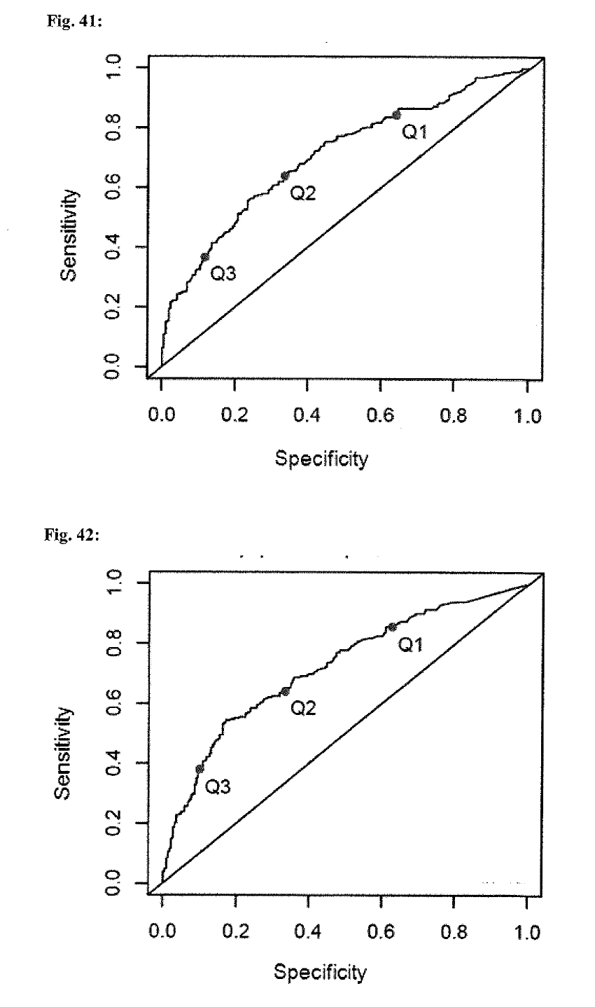

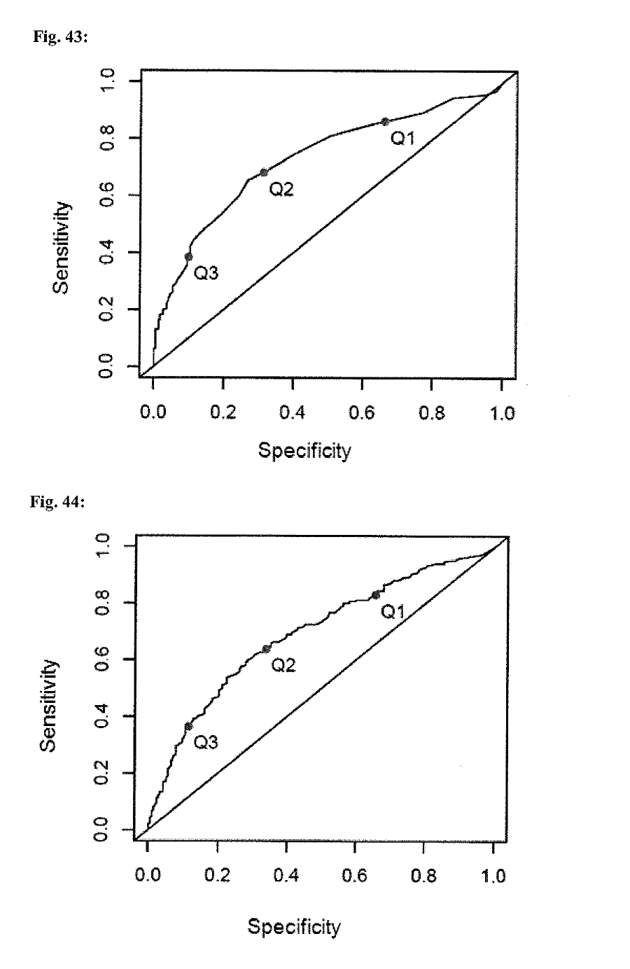

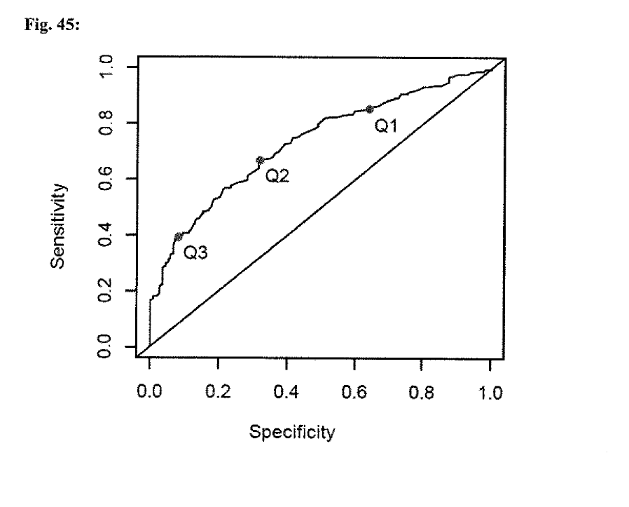

### Tables

TABLE 1
Criteria for serious condition (in BANC study and BANC study III)

| | |
|---|---|
| Cardiovascular | |
| Congestive heart failure | all new, if symptomatic, or need of further medication, or |
| Right heart failure | intervention <24 h |
| Acute coronary syndrome | all <48 h |
| Aneurysms | all new, if symptomatic |
| Peripheral arterial disease | all new, if Fontaine III + IV |
| Peri-/myocarditis | all new, if signs of heart failure, or arrhythmia, or tamponade |
| Pulmonary | |
| Pulmonary Embolism/ | all new, if confirmed |
| DVT | |
| Interstitial Pulmonary | all new, if symptomatic, or complications requiring intervention |
| Disease | <24 h |
| COPD, sleep apnoea, | |
| Hypoventilation, Pleural | |
| effusion | |
| Respiratory failure | |
| Abdominal | |
| Ascites | all new, or SBP |
| Hernia | all new, if symptomatic, and complications |
| Gastrointestinal | all confirmed bleedings |
| hemorrhagic | |
| Acute hepatic failure | all |
| Acute abdomen | all |
| Peptic ulcer | all new, or requiring intervention <24 h |
| Chronic inflammatory | all new, or complications requiring intervention <24 h |
| bowel disease | |
| Gastroenteritis | See in section infectious |
| Neurological | |
| Cerebrovascular - ischemic | all with onset <24 h |
| Cerebrovascular - | all with onset <24 h, or pre-existing with new symptoms |
| hemorrhagic | |
| Subdural hematoma, | |
| Brain abscess | all with onset <24 h |
| Meningitis | all |
| Brain tumour | all new |
| Radicular Compression | all with functional disability |
| Epilepsy | all seizures requiring therapy change, all unprovoked seizures |
| Hydrocephalus | all new, or preexisting with new symptoms |
| Radiculitis | all Guillain-Barre Syndrome, or Miller-Fisher Syndrome |
| Increased Intracranial | all new |
| pressure | |
| Wernicke encephalopathy | |
| Infectious | all proven bacterial, viral or fungal |
| | all with >1 criteria for SIRS |
| Pneumonia | all new, or requiring intervention <24 h |
| Gastroenteritis/Diarrhoea | all with severe symptoms (i.e. >1 SIRS criteria or signs of |
| | dehydration (see below)) |
| Urinary tract infection | all with >1 criteria for SIRS, or if acute urinary retention present |
| Cholecystitis | all new |
| Diverticulitis | all new |
| Necrotising fasciitis | all new |
| Sepsis | all |
| Septic Arthritis | all |
| Endocrine, nutritional and metabolic diseases | |
| Hyponatremia/Hypernatremia | all with Na <131 mmol/l except pseudohyponatremia; or |
| | Na>154 mmol/l |
| Hypokalemia/Hyperkalemia | all with K <3.1 mmol/l; or >5.9 mmol/l |
| Hypocalcemia/Hypercalcemia | all with Ca <2.10, or >2.65 mmol/l (corrected for serum |
| | albumin 35-52 g/L) |
| Dehydration | all with clinical findings, such as positive Schellong test or |
| | empty jugular veins, or typical lab findings (elevated urea, |
| | protein or hematocrit) |
| Hypoglycemia | all symptomatic, and glucose <2.8 mmol/L |
| Diabetes mellitus | all new, or, ketoacidosis, severe dehydration, altered mental |
| | state |
| M. Addison | all with cortisol >200, or stimulated cortisol <500 ng/L |
| Acidosis | all with pH <7.2 and related symptoms |
| Hypothyroidism | all new and symptomatic |
| Hyperthyroidism | all new and symptomatic |
| Blood, Blood-forming organs, Immune system | |
| Hemolysis | all new |
| Anemia | all Hb <100 g/l, if symptomatic |
| Bone marrow disorders | all new lymphoproliferative disease, leucemia, myelodysplastic |
| | syndrome |
| Nephrology/Urology | |
| Acute urinary retention | all |
| chronic renal failure | all complications (electrolyte disorders, metabolic acidosis, |
| | uremia) |
| Acute renal failure | all rise of serum creatinine >44 mmol/L or anuria >24 h |
| Poisoning/Intoxication | all potentially life-threatening |
| Musculoskeletal system and connective tissue | |
| Acute rheumatological | all new and symptomatic |
| diseases | |
| Neoplasms | all new, or acute complications, or potentially life-threatening, |
| | all deaths within 30 d |
| Mental and behavioral | |
| Delirium | All new (onset <24 h) according to DSM IV classification |
| Others | |
| Rhabdomyolysis | all CK >5000 U/l |
| Fractures | all requiring surgical intervention |
| Medication Side Effects | all new, if serious outcome of any kind |
| Graft versus host reaction | all new |
| Other iatrogenic | all new, if serious outcome of any kind |

TABLE 2
Baseline characteristics of patients (BANC study)

| Characteristics | n (%) | |
|---|---|---|
| Total number | 438 | |
| Male | 154 | (35%) |
| Female | 284 | (65%) |
| Age, Median (Range) | 80 | (22-101) |
| Age ≧65 | 375 | (85.6%) |
| ESI category | | |
| 2 | 10 | (2.3%) |
| 3 | 428 | (97.7%) |
| Comorbidities; Median (Range) | 5 | (0-11) |
| Charlson Comorbidity Index; Median (Range) | 2 | (0-13) |
| Number of medicaments taking daily, Median (Range) | 5 | (0-17) |
| ADL after Katz; Median | 6 | |
| Katz Index <6 | 190 | (43.4%) |

TABLE 3
Prediction of death within 30-days (BANC study)

| | Model χ² | p-value | C-index |
|---|---|---|---|
| Univariate Model | | | |
| PRX-4 | 22.24 | <0.00001 | 0.749 |
| Copeptin | 16.98 | 0.00004 | 0.724 |
| PCT | 11.41 | 0.00073 | 0.705 |
| MR-proANP | 7.56 | 0.00596 | 0.669 |
| Multivariate Model | | | |
| PRX-4, Copeptin | 28.22 | <0.00001 | 0.783 |
| PRX-4, MR-proANP | 21.14 | 0.00003 | 0.757 |
| PRX-4, PCT | 20.81 | 0.00003 | 0.738 |
| PRX-4, Urea | 30.67 | <0.00001 | 0.785 |
| PRX-4, Albumin | 33.47 | <0.00001 | 0.777 |
| PRX-4, CRP | 23.48 | 0.00001 | 0.748 |
| Copeptin, MR-proANP | 15.43 | 0.00045 | 0.707 |
| Copeptin, PCT | 20.82 | 0.00003 | 0.739 |
| Copeptin, Albumin | 38.53 | <0.00001 | 0.808 |
| Copeptin, CRP | 22.28 | 0.00001 | 0.752 |
| Copeptin, Urea | 21.82 | 0.00002 | 0.749 |
| PCT, MR-proANP | 14.34 | 0.00077 | 0.720 |
| PCT, Albumin | 27.21 | <0.00001 | 0.761 |
| PCT, Urea | 20.23 | 0.00004 | 0.741 |
| PCT, CRP | 13.89 | 0.00096 | 0.708 |
| MR-proANP, Albumin | 28.67 | <0.00001 | 0.774 |
| MR-proANP, CRP | 14.29 | 0.00079 | 0.720 |
| MR-proANP, Urea | 16.56 | 0.00025 | 0.716 |
| PRX-4, Copeptin, Katz | 37.07 | <0.00001 | 0.807 |
| ADL | | | |
| PRX-4, Copeptin, Charlson | 30.78 | <0.00001 | 0.791 |
| Comorbidity Index | | | |
| PRX-4, Copeptin, age, | 32.87 | <0.00001 | 0.788 |
| gender | | | |

TABLE 4
Prediction of a serious condition within 30-days (BANC study)

| Model | Model χ² | p-value | C-index |
|---|---|---|---|
| PCT | 38.23 | <0.00001 | 0.666 |
| Copeptin | 21.51 | <0.00001 | 0.641 |
| PRX-4 | 11.16 | 0.00083 | 0.589 |
| MR-proANP | 8.57 | 0.00342 | 0.607 |
| PCT, Copeptin | 44.12 | <0.00001 | 0.679 |
| PCT, Urea | 45.57 | <0.00001 | 0.669 |
| PCT, PRX-4 | 38.87 | <0.00001 | 0.665 |
| PCT, MR-proANP | 37.77 | <0.00001 | 0.659 |
| PCT, CRP | 40.11 | <0.00001 | 0.661 |
| PCT, Albumin | 39.20 | <0.00001 | 0.664 |
| Copeptin, PRX-4 | 26.63 | <0.00001 | 0.645 |
| Copeptin, MR-proANP | 21.12 | 0.00003 | 0.637 |
| Copeptin, Urea | 34.21 | <0.00001 | 0.661 |
| Copeptin, CRP | 34.88 | <0.00001 | 0.653 |
| Copeptin, Albumin | 29.10 | <0.00001 | 0.632 |
| PRX-4, Urea | 33.13 | <0.00001 | 0.646 |
| PRX-4, CRP | 21.38 | 0.00002 | 0.625 |
| PRX-4, MR-proANP | 17.12 | 0.00019 | 0.613 |
| PRX-4, Albumin | 15.51 | 0.00043 | 0.591 |
| MR-proANP, Urea | 27.48 | <0.00001 | 0.633 |
| MR-proANP, CRP | 22.65 | 0.00001 | 0.625 |
| MR-proANP, Albumin | 16.68 | 0.00024 | 0.606 |

TABLE 5
Prediction of admission to the ICU
(stay on ICU ≧10 days) within 30-days (BANC study)

| Model | Model χ² | p-value | C-index |
|---|---|---|---|
| PCT | 20.01 | 0.00001 | 0.615 |
| PRX-4 | 9.20 | 0.00242 | 0.587 |
| Copeptin | 7.66 | 0.00566 | 0.574 |
| MR-proANP | 4.72 | 0.02977 | 0.570 |
| PCT, PRX-4 | 21.37 | 0.00002 | 0.617 |
| PCT, Copeptin | 21.06 | 0.00003 | 0.611 |
| PCT, MR-proANP | 20.78 | 0.00003 | 0.609 |
| PCT, CRP | 33.24 | <0.00001 | 0.654 |
| PCT, Urea | 25.20 | <0.00001 | 0.624 |
| PCT, Albumin | 24.39 | 0.00001 | 0.623 |
| PRX-4, Copeptin | 15.55 | 0.00042 | 0.613 |
| PRX-4, MR-proANP | 13.51 | 0.00116 | 0.601 |
| PRX-4, CRP | 28.50 | <0.00001 | 0.645 |
| PRX-4, Urea | 22.02 | 0.00002 | 0.617 |
| PRX-4, Albumin | 15.21 | 0.00050 | 0.605 |
| Copeptin, MR-proANP | 8.50 | 0.01428 | 0.573 |
| Copeptin, CRP | 31.23 | <0.00001 | 0.652 |
| Copeptin, Albumin | 19.45 | 0.00006 | 0.625 |
| Copeptin, Urea | 17.08 | 0.00020 | 0.598 |
| MR-proANP, CRP | 31.21 | <0.00001 | 0.653 |
| MR-proANP, Albumin | 17.58 | 0.00015 | 0.610 |
| MR-proANP, Urea | 16.86 | 0.00022 | 0.597 |

TABLE 6
Prediction of hospitalization (≧ 30 days) (BANC study)

| Model | Model χ² | p-value | C-index |
|---|---|---|---|
| Copeptin | 7.94 | 0.00485 | 0.584 |
| MR-proANP | 3.09 | 0.07869 | 0.553 |
| PCT | 2.15 | 0.14297 | 0.566 |
| PRX-4 | 1.32 | 0.25104 | 0.541 |
| Copeptin, PRX-4 | 8.74 | 0.01263 | 0.583 |
| Copeptin, MR-proANP | 8.43 | 0.01479 | 0.573 |
| Copeptin, PCT | 7.38 | 0.02502 | 0.568 |
| Copeptin, Urea | 10.54 | 0.00513 | 0.580 |
| Copeptin, CRP | 10.13 | 0.00631 | 0.583 |
| Copeptin, Albumin | 8.56 | 0.01382 | 0.576 |
| MR-proANP, PRX-4 | 4.28 | 0.11737 | 0.544 |
| MR-proANP, PCT | 3.43 | 0.18012 | 0.548 |
| MR-proANP, Urea | 8.73 | 0.01273 | 0.584 |
| MR-proANP, CRP | 6.24 | 0.04407 | 0.561 |
| MR-proANP, Albumin | 3.41 | 0.18182 | 0.539 |
| PCT, PRX-4 | 2.37 | 0.30625 | 0.538 |
| PCT, Urea | 7.95 | 0.01880 | 0.574 |
| PCT, CRP | 4.58 | 0.10151 | 0.550 |
| PCT, Albumin | 3.37 | 0.18537 | 0.544 |
| PRX-4, Urea | 8.78 | 0.01242 | 0.573 |
| PRX-4, CRP | 4.59 | 0.10095 | 0.551 |
| PRX-4, Albumin | 2.67 | 0.26345 | 0.531 |

TABLE 7
Prediction of death within 6 months (BANC study)

| Model | Model χ² | p-value | C-index |
|---|---|---|---|
| PRX-4 | 22.46 | <0.00001 | 0.703 |
| Copeptin | 11.25 | 0.00080 | 0.660 |
| PCT | 7.15 | 0.00748 | 0.625 |
| MR-proANP | 1.45 | 0.22847 | 0.574 |
| PRX-4, Copeptin | 25.20 | <0.00001 | 0.710 |
| PRX-4, PCT | 20.39 | 0.00004 | 0.690 |
| PRX-4, MR-proANP | 18.55 | 0.00009 | 0.683 |
| PRX-4, Albumin | 37.04 | <0.00001 | 0.739 |
| PRX-4, Urea | 28.15 | <0.00001 | 0.717 |
| PRX-4, CRP | 25.02 | <0.00001 | 0.711 |
| Copeptin, PCT | 13.69 | 0.00107 | 0.644 |
| Copeptin, MR-proANP | 9.82 | 0.00737 | 0.638 |
| Copeptin, Albumin | 37.81 | <0.00001 | 0.754 |
| Copeptin, CRP | 19.94 | 0.00005 | 0.684 |
| Copeptin, Urea | 15.36 | 0.00046 | 0.661 |
| PCT, MR-proANP | 7.03 | 0.02970 | 0.599 |
| PCT, Albumin | 29.47 | <0.00001 | 0.719 |
| PCT, CRP | 13.87 | 0.00097 | 0.659 |
| PCT, Urea | 14.26 | 0.00080 | 0.644 |
| MR-proANP, Albumin | 29.21 | <0.00001 | 0.718 |
| MR-proANP, Urea | 11.51 | 0.00317 | 0.631 |
| MR-proANP, CRP | 12.90 | 0.00158 | 0.653 |

TABLE 8
Sensitivities and Specificities (in %) for different MR-proANP
cut-off values measured on admission in patients with NSC for the
prediction of death within 30 days/6 months and prediction of serious
condition including death within 30 days (BANC study)

| | | | | | Prediction of serious | |
|---|---|---|---|---|---|---|
| | Prediction of death | | Prediction of death | | condition indluding | |
| Cut | within 30 days | | within 6 months | | death within 30 days | |
| off | Sensi- | Speci- | Sensi- | Speci- | Sensi- | Speci- |
| (in | tivity | ficity | tivity | ficity | tivity | ficity |
| pmol/L) | (%) | (%) | (%) | (%) | (%) | (%) |
| 81.1 | 92.0 | 31.3 | 80.0 | 31.0 | 73.8 | 34.8 |
| 133.7 | 80.0 | 42.2 | 62.5 | 41.3 | 58.3 | 56.7 |
| 142 | 76.0 | 51.7 | 60.0 | 51.5 | 57.5 | 60.1 |
| 184.2 | 72.0 | 61.8 | 55.0 | 61.4 | 50.0 | 73.0 |
| 326.8 | 40.0 | 81.7 | 30.0 | 81.5 | 20.4 | 89.3 |
| 428.1 | 24.0 | 90.8 | 15.0 | 90.5 | 13.3 | 93.8 |

TABLE 9
Sensitivities and Specificities (in %) for different Copeptin cut-
off values measured on admission in patients with NSC for the prediction
of death within 30 days/6 months and prediction of serious condition
including death within 30 days (BANC study)

| | | | | | Prediction of serious | |
|---|---|---|---|---|---|---|
| | Prediction of death | | Prediction of death | | condition including | |
| Cut | within 30 days | | within 6 months | | death within 30 days | |
| off | Sensi- | Speci- | Sensi- | Speci- | Sensi- | Speci- |
| (in | tivity | ficity | tivity | ficity | tivity | ficity |
| pmol/L) | (%) | (%) | (%) | (%) | (%) | (%) |
| 4.7 | 96.2 | 27.3 | 92.7 | 27.9 | 79.0 | 31.5 |
| 10.2 | 88.5 | 42.5 | 80.5 | 42.9 | 68.3 | 51.7 |
| 19.2 | 80.8 | 57.5 | 65.9 | 57.4 | 56.0 | 69.7 |
| 30.9 | 73.1 | 69.1 | 58.5 | 69.2 | 42.0 | 78.1 |
| 41.5 | 53.8 | 77.5 | 51.2 | 75.0 | 35.4 | 83.1 |
| 81.5 | 26.9 | 87.3 | 22.0 | 87.4 | 18.5 | 92.7 |

TABLE 10
Sensitivities and Specificities (in %) for different PCT cut-off
values measured on admission in patients with NSC for the prediction
of death within 30 days/6 months and prediction of serious condition
including death within 30 days (BANC study)

| | | | | | Prediction of serious | |
|---|---|---|---|---|---|---|
| | Prediction of death | | Prediction of death | | condition including | |
| Cut | within 30 days | | within 6 months | | death within 30 days | |
| off | Sensi- | Speci- | Sensi- | Speci- | Sensi- | Speci- |
| (in | tivity | ficity | tivity | ficity | tivity | ficity |
| ng/mL) | (%) | (%) | (%) | (%) | (%) | (%) |
| 0.036 | 84.6 | 32.1 | 75.6 | 31.7 | 74.0 | 37.9 |
| 0.047 | 80.8 | 44.5 | 70.7 | 44.4 | 66.9 | 56.5 |
| 0.145 | 69.2 | 77.9 | 53.7 | 78.0 | 35.0 | 88.7 |
| 0.181 | 53.8 | 80.7 | 41.5 | 80.7 | 31.0 | 91.0 |
| 0.250 | 46.2 | 84.7 | 36.6 | 84.9 | 25.2 | 93.8 |
| 0.330 | 42.3 | 90.3 | 29.3 | 90.2 | 17.8 | 96.6 |
| 0.500 | 26.9 | 92.6 | 19.5 | 92.6 | 13.2 | 97.2 |

TABLE 11
Sensitivities and Specificities (in %) for different PRX-4 cut-
off values measured on admission in patients with NSC for the
prediction of death within 30 days/6 months and prediction of
serious condition including death within 30 days (BANC study)

| | | | | | Prediction of serious | |
|---|---|---|---|---|---|---|
| | Prediction of death | | Prediction of death | | condition including | |
| Cut | within 30 days | | within 6 months | | death within 30 days | |
| off | Sensi- | Speci- | Sensi- | Speci- | Sensi- | Speci- |
| (in | tivity | ficity | tivity | ficity | tivity | ficity |
| U/mL) | (%) | (%) | (%) | (%) | (%) | (%) |
| 3.11 | 92.6 | 28.5 | 90.5 | 29.1 | 77.6 | 33.7 |
| 3.84 | 85.2 | 42.7 | 78.6 | 43.0 | 63.3 | 46.6 |
| 4.57 | 81.5 | 54.0 | 73.8 | 54.6 | 52.2 | 57.3 |
| 6.03 | 74.1 | 69.7 | 69.0 | 70.9 | 39.6 | 74.7 |
| 7.97 | 66.7 | 80.8 | 57.1 | 81.6 | 27.3 | 84.8 |
| 10.94 | 40.7 | 90.4 | 33.3 | 90.8 | 17.1 | 94.9 |

TABLE 12
Baseline characteristics of patients (BANC study III)

| Characteristics | n (%) | |
|---|---|---|
| Total number | 504 | |
| Male | 196 | (38.9%) |
| Female | 308 | (61.1%) |
| Age, Median (Range) | 82 | (75-87) |
| Age ≧65 | 448 | (88.9%) |
| Charlson Comorbidity Index; Median (IQR) | 2 | (0-3) |
| ADL after Katz; Median | 5 | |
| Katz Index <6 | 276 | (54.5%) |
| Disposition | | |
| Discharged | 68 | (13.5%) |
| Transfer to geriatric hospital | 145 | (28.8%) |
| Admission to tertiary care hospital | 291 | (57.7%) |

TABLE 13
Sensitivities and Specificities (in %) for different MR-proADM
cut-off values measured on admission in patients with NSC
for the prediction of death within 30 days and the prediction
of serious condition including death within 30 days (BANC
study III)

| | | | Prediction of serious | |
|---|---|---|---|---|
| | Prediction of death | | condition including | |
| | within 30 days | | death within 30 days | |
| Cut off | Sensitivity | | Sensitivity | |
| (in nmol/L) | (%) | Specificity (%) | (%) | Specificity (%) |
| 0.55 | 100.0 | 12.7 | 93.7 | 16.6 |
| 0.75 | 93.9 | 26.5 | 85.2 | 35.9 |
| 0.80 | 93.9 | 32.1 | 82.3 | 45.7 |
| 1.00 | 84.8 | 50.4 | 67.5 | 65.0 |
| 1.50 | 54.5 | 75.2 | 40.6 | 90.1 |
| 1.94 | 48.5 | 86.0 | 27.7 | 96.4 |
| 2.95 | 30.3 | 95.9 | 11.1 | 100 |

TABLE 14
Prediction of death within 30-days (BANC study III)

| | Model χ² | p-value | C-index |
|---|---|---|---|
| Univariate Model | | | |
| PRX-4 | 18.73 | <0.0001 | 0.719 |
| Copeptin | 20.81 | <0.0001 | 0.723 |
| PCT | 8.67 | <0.01 | 0.69 |
| MR-proANP | 22.37 | <0.0001 | 0.697 |
| MR-proADM | 29.74 | <0.0001 | 0.732 |
| Multivariate Model | | | |
| PRX-4, Copeptin | 30.41 | <0.0001 | 0.76 |
| PRX-4, MR-proANP | 34.2 | <0.0001 | 0.769 |
| PRX-4, PCT | 20.4 | <0.0001 | 0.727 |
| PRX-4, MR-proADM | 35.68 | <0.0001 | 0.769 |
| PRX-4, CRP | 20.1 | <0.0001 | 0.715 |
| PRX-4, Urea | 28.01 | <0.0001 | 0.767 |
| PRX-4, Albumin | 33.43 | <0.0001 | 0.774 |
| Copeptin, MR-proANP | 29.31 | <0.0001 | 0.725 |
| Copeptin, PCT | 22.08 | <0.0001 | 0.719 |
| Copeptin, MR-proADM | 31.55 | <0.0001 | 0.738 |
| Copeptin, Urea | 22.75 | <0.0001 | 0.725 |
| Copeptin, CRP | 25.08 | <0.0001 | 0.737 |
| Copeptin, Albumin | 43.41 | <0.0001 | 0.807 |
| PCT, MR-proANP | 24.98 | <0.0001 | 0.723 |
| PCT, MR-proADM | 29.68 | <0.0001 | 0.717 |
| PCT, CRP | 14.11 | <0.0001 | 0.681 |
| PCT, Urea | 17.43 | <0.0001 | 0.71 |
| PCT, Albumin | 29.76 | <0.0001 | 0.764 |
| MR-proANP, MR-proADM | 30.73 | <0.0001 | 0.727 |
| MR-proANP, Urea | 25.15 | <0.0001 | 0.708 |
| MR-proANP, CRP | 30.91 | <0.0001 | 0.76 |
| MR-proANP, Albumin | 41.71 | <0.0001 | 0.805 |
| MR-proADM, Urea | 29.81 | <0.0001 | 0.732 |
| MR-proADM, CRP | 31.63 | <0.0001 | 0.747 |
| MR-proADM, Albumin | 43.34 | <0.0001 | 0.804 |
| PRX-4, Copeptin, Katz ADL | 52.15 | <0.0001 | 0.819 |
| PRX-4, Copeptin, Charlson | 37.92 | <0.0001 | 0.744 |
| Comorbidity Index | | | |
| PRX-4, Copeptin, age, | 33.77 | <0.0001 | 0.76 |
| gender | | | |

TABLE 15
Prediction of admission to the ICU
(stay on ICU ≧10 days) within 30-days (BANC study III)

| | Model χ² | p-value | C-index |
|---|---|---|---|
| Univariate Model | | | |
| PRX-4 | 21.88 | <0.0001 | 0.623 |
| Copeptin | 18.31 | <0.0001 | 0.613 |
| PCT | 20.21 | <0.0001 | 0.619 |
| MR-proANP | 14.84 | <0.0001 | 0.598 |
| MR-proADM | 18.2 | <0.0001 | 0.601 |
| Multivariate Model | | | |
| PRX-4, Copeptin | 29.54 | <0.0001 | 0.636 |
| PRX-4, MR-proANP | 30.18 | <0.0001 | 0.63 |
| PRX-4, PCT | 27.74 | <0.0001 | 0.629 |
| PRX-4, MR-proADM | 28.65 | <0.0001 | 0.627 |
| PRX-4, Albumin | 23.77 | <0.0001 | 0.622 |
| PRX-4, Urea | 24.05 | <0.0001 | 0.615 |
| PRX-4, CRP | 25.28 | <0.0001 | 0.624 |
| Copeptin, MR-proANP | 21.79 | <0.0001 | 0.618 |
| Copeptin, PCT | 27.33 | <0.0001 | 0.632 |
| Copeptin, MR-proADM | 22.07 | <0.0001 | 0.615 |
| Copeptin, Urea | 19.28 | <0.0001 | 0.61 |
| Copeptin, Albumin | 22.41 | <0.0001 | 0.62 |
| Copeptin, CRP | 24.58 | <0.0001 | 0.62 |
| PCT, MR-proANP | 27.72 | <0.0001 | 0.628 |
| PCT, MR-proADM | 24.13 | <0.0001 | 0.612 |
| PCT, Urea | 20.62 | <0.0001 | 0.608 |
| PCT, Albumin | 23.09 | <0.0001 | 0.618 |
| PCT, CRP | 23.94 | <0.0001 | 0.615 |
| MR-proANP, MR-proADM | 19.5 | <0.0001 | 0.605 |
| MR-proANP, Urea | 15.1 | <0.001 | 0.594 |
| MR-proANP, Albumin | 18.99 | <0.0001 | 0.604 |
| MR-proANP, CRP | 26.76 | <0.0001 | 0.625 |
| MR-proADM, Urea | 18.26 | <0.001 | 0.597 |
| MR-proADM, Albumin | 20.68 | <0.0001 | 0.605 |
| MR-proADM, CRP | 23.55 | <0.0001 | 0.612 |

TABLE 16
Prediction of serious condition within 30-days (BANC study III)

| | Model χ² | p-value | C-index |
|---|---|---|---|
| Univariate Model | | | |
| PRX-4 | 54.08 | <0.0001 | 0.694 |
| Copeptin | 74.17 | <0.0001 | 0.722 |
| PCT | 68.94 | <0.0001 | 0.73 |
| MR-proANP | 68.26 | <0.0001 | 0.706 |
| MR-proADM | 87.74 | <0.0001 | 0.732 |
| Multivariate Model | | | |
| PRX-4, Copeptin | 94.12 | <0.0001 | 0.745 |
| PRX-4, MR-proANP | 101.95 | <0.0001 | 0.752 |
| PRX-4, PCT | 84.07 | <0.0001 | 0.738 |
| PRX-4, MR-proADM | 102.4 | <0.0001 | 0.754 |
| PRX-4, Albumin | 70.24 | <0.0001 | 0.714 |
| PRX-4, Urea | 110.81 | <0.0001 | 0.762 |
| PRX-4, CRP | 79.81 | <0.0001 | 0.726 |
| Copeptin, MR-proANP | 94.65 | <0.0001 | 0.737 |
| Copeptin, PCT | 98.24 | <0.0001 | 0.75 |
| Copeptin, MR-proADM | 99.5 | <0.0001 | 0.742 |
| Copeptin, Urea | 87.65 | <0.0001 | 0.728 |
| Copeptin, Albumin | 101.63 | <0.0001 | 0.754 |
| Copeptin, CRP | 107.71 | <0.0001 | 0.762 |
| PCT, MR-proANP | 107.89 | <0.0001 | 0.758 |
| PCT, MR-proADM | 100.63 | <0.0001 | 0.742 |
| PCT, Urea | 101.27 | <0.0001 | 0.751 |
| PCT, Albumin | 85.56 | <0.0001 | 0.741 |
| PCT, CRP | 90.07 | <0.0001 | 0.735 |
| MR-proANP, MR-proADM | 95.69 | <0.0001 | 0.743 |
| MR-proANP, Urea | 93.98 | <0.0001 | 0.737 |
| MR-proANP, Albumin | 91.81 | <0.0001 | 0.745 |
| MR-proANP, CRP | 125.51 | <0.0001 | 0.777 |
| MR-proADM, Urea | 99.03 | <0.0001 | 0.738 |
| MR-proADM, Albumin | 103.45 | <0.0001 | 0.757 |
| MR-proADM, CRP | 111.13 | <0.0001 | 0.758 |

1. A method for the risk assessment or the prognosis of an outcome or the stratification of patients with non-specific complaints,
the method comprising the steps of:
providing a sample from a bodily fluid from said patient,
determining in the sample the level of a marker peptide selected from the group of proANP, proBNP, proAVP, pro ADM, proET-1, PCT, PRX-4 or fragments thereof comprising at least 12 amino acids in length, and
correlating said marker peptide level to the risk of acquiring a serious condition and/or death or to the prognosis of an outcome in a patient with non-specific complaints.

2. A method according to claim 1, wherein serious conditions is defined as potentially life-threatening or requiring early intervention to prevent health status deterioration.

3. A method according to claim 1 wherein the risk assessment or the prognosis or the stratification relates to the risk of getting a serious condition or patients are stratified into either a group of patients likely getting a serious condition and/or death or into a group of patients which do not likely get a serious condition and/or death.

4. A method according to claim 3, wherein the risk assessment relates to the risk of getting a serious condition within 1 year, more preferred within 6 months, even more preferred within 90 days, even more preferred within 60 days, most preferred within 30 days.

5. A method according to claim 3, wherein the serious condition is selected from the group comprising death, hospitalisation or admission to ICU.

6. A method according to claim 1, wherein patient stratification relates to the management of a patient including the decision for admission to hospital or intensive care unit, the decision for relocation of the patient to a specialized hospital or a specialized hospital unit, the decision for relocation of the patient to a specialized hospital or a specialized hospital unit, the evaluation for an early discharge from the intensive care unit or hospital or the allocation of resources (e.g. physician and/or
nursing staff, diagnostics, therapeutics) or the stratification of the patients relates to the severity of their condition.

7. A method according to claim 1, wherein the level of at least two marker peptides is determined.

8. A method according to claim 1, wherein the level of at least one more marker selected from the group comprising C-reactive protein, creatinine, albumin, urea, glomerular filtration rate, count of white blood cell, troponin, myeloperoxidase, neopterin, GDF-15, ST2, cystatin-C is determined.

9. A method according to claim 1, wherein the level of at least one marker peptide is combined with at least one parameter selected from the group comprising age, gender, Charlson Comorbidity Index (CCI), Katz ADL.

10. A method according to claim 1, wherein the level of the precursor fragment MR-proANP is determined.

11. A method according to claim 1, wherein the level of the precursor fragment MR-proADM is determined.

12. A method according to claim 1, wherein the level of the precursor fragment Copeptin is determined.

13. A method according to claim 1, wherein the precursor fragment CT-proET-1 is determined.

14. A method according to claim 1, wherein the precursor fragment NT-proBNP is determined.

15. A method according to claim 1, wherein PCT fragment 1 to 116 or 2 to 116 or 3 to 116 is determined.


SEQUENCE LISTING (20 sequences)

SEQ ID NO: 1 - protein, 153 aa, Homo sapiens [sequence omitted]

SEQ ID NO: 2 - protein, 126 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 3 - protein, 98 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPR

SEQ ID NO: 4 - protein, 38 aa, Homo sapiens
PEVPPWTGEVSPAQRDGGALGRGPWDSSDRSALLKSKL

SEQ ID NO: 5 - protein, 185 aa, Homo sapiens
MKLVSVALMYLGSLAFLGADTARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 6 - protein, 164 aa, Homo sapiens
ARLDVASEFRKKWNKWALSRGKRELRMSSSYPTGLADVKAGPAQTLIRPQDMKGASRSPEDSSPDAARIRVKRYRQSMNNFQGLRSFGCRFGTCTVQKLAHQIYQFTDKDKDNVAPRSKISPQGYGRRRRRSLPEAGPGRTLVSSKPQAHGAPAPPSGSAPHFL

SEQ ID NO: 7 - protein, 48 aa, Homo sapiens
ELRMSSSYPTGLADVKAGPAQTLIRPQDMKGASRSPEDSSPDAARIRV

SEQ ID NO: 8 - protein, 164 aa, Homo sapiens
MPDTMLPACFLGLLAFSSACYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 9 - protein, 145 aa, Homo sapiens
CYFQNCPRGGKRAMSDLELRQCLPCGPGGKGRCFGPSICCADELGCFVGTAEALRCQEENYLPSPCQSGQKACGSGGRCAAFGVCCNDESCVTEPECREGFHRRARASDRSNATQLDGPAGALLLRLVQLAGAPEPFEPAQPDAY

SEQ ID NO: 10 - protein, 39 aa, Homo sapiens
ASDRSNATQLDGPAGALLLRLVQLAGAPEPFEPAQPDAY

SEQ ID NO: 11 - protein, 93 aa, Homo sapiens
AMSDLELRQCLPCGPGGKGRCFGPSICCADELGCFVGTAEALRCQEENYLPSPCQSGQKACGSGGRCAAFGVCCNDESCVTEPECREGFHRRA

SEQ ID NO: 12 - protein, 212 aa, Homo sapiens
MDYLLMIFSLLFVACQGAPETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 13 - protein, 195 aa, Homo sapiens
APETAVLGAELSAVGENGGEKPTPSPPWRLRRSKRCSCSSLMDKECVYFCHLDIIWVNTPEHVVPYGLGSPRSKRALENLLPTKATDRENRCQCASQKDKKCWNFCQAGKELRAEDIMEKDWNNHKKGKDCSKLGKKCIYQQLVRGRKIRRSSEEHLRQTRSETMRNSVKSSFHDPKLKGKPSRERYVTHNRAHW

SEQ ID NO: 14 - protein, 45 aa, Homo sapiens
RSSEEHLRQTRSETMRNSVKSSFHDPKLKGKPSRERYVTHNRAHW

SEQ ID NO: 15 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 16 - protein, 108 aa, Homo sapiens
HPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 17 - protein, 76 aa, Homo sapiens
HPLGSPGSASDLETSGLQEQRNHLQGKLSELQVEQTSLEPLQESPRPTGVWKSREVATEGIRGHRKMVLYTLRAPR

SEQ ID NO: 18 - protein, 32 aa, Homo sapiens
SPKMVQGSGCFGRKMDRISSSSGLGCKVLRRH

SEQ ID NO: 19 - protein, 116 aa, Homo sapiens
APFRSALESSPADPATLSEDEARLLLAALVQDYVQMKASELEQEQEREGSSLDSPRSKRCGNLSTCMLGTYTQDFNKFHTFPQTAIGVGAPGKKRDMSSDLERDHRPHVSMPQNAN

SEQ ID NO: 20 - protein, 271 aa, Homo sapiens
MEALPLLAATTPDHGRHRRLLLLPLLLFLLPAGAVQGWETEERPRTREEECHFYAGGQVYPGEASRVSVADHSLHLSKAKISKPAPYWEGTAVIDGEFKELKLTDYRGKYLVFFFYPLDFTFVCPTEIIAFGDRLEEFRSINTEVVACSVDSQFTHLAWINTPRRQGGLGPIRIPLLSDLTHQISKDYGVYLEDSGHTLRGLFIIDDKGILRQITLNDLPVGRSVDETLRLVQAFQYTDKHGEVCPAGWKPGSETIIPDPAGKLKYFDKLN